SEQUENCE LISTING (6 sequences)

SEQ ID NO: 1 - DNA, 2650 bp, Mus musculus
  CDS (280)...(1422)
cacgcgtccgattaattggatttcattcactgggtaggaacaaaactgggcaccttcattcagagagagagattcattgactcggagagtgatctggtgcagagggaccaccgacttgacttctgtgtcgctttccctaaccgctagcctcggcttgggaaaggcgaggcggaatcaaaccccgctccgagagcgggagcttcgcgcagcgtgctcggcctatgcctgcctcgaggggcgtctgctaggcaccccgccttctcctgcagctcgacccccatgatagatacgctcagacccgtgcccttcgcgtcggaaatggcgatctgcaagacggtgtcgtggctcaacgagcagctggagctgggcaacgaacggcttctgctgatggactgccgaccacaggagctgtacgagtcgtcacacatcgaatctgccattaatgtggccatccccggcatcatgctgcggcgtctgcagaagggcaacctgcccgtgcgtgcgctcttcacgcgctgcgaggaccgggaccgctttaccaggcgctgcggcaccgacaccgtggtgctgtacgacgagaatagcagcgactggaatgagaacactggtggagagtcggtcctcgggctgctgctcaagaaactcaaagacgagggctgccgggcgttctacctggaaggtggcttcagtaagttccaggccgagttcgccctgcactgcgagaccaatctagacggctcgtgcagcagcagttccccgcctttgccagtgctggggctcgggggcctgcggatcagctcggactcttcctcggacattgagtctgaccttgaccgagaccccaatagtgcaacggactctgatggcagcccgctgtccaacagccagccttccttcccggtggagattttgcccttcctttacctgggctgtgccaaggactcgaccaacttggacgtgttggaagagtttggcatcaagtacatcttgaatgtcacccccaatttgcccaatctgtttgagaatgcgggcgagttcaaatacaagcaaattcctatctcggatcactggagccaaaacctgtcccagtttttccctgaggccatttctttcatagatgaagcccgaggcaaaaactgtggtgtcctggtgcattgcttggcaggtatcagccgctctgtcaccgtgacagtggcgtacctcatgcagaagctcaacctgtccatgaacgatgcttacgacattgttaagatgaagaagtccaacatctcccccaacttcaacttcatgggccagctgcttgacttcgaaaggaccctgggactgagcagcccttgtgacaaccgtgtccccactccgcagctgtacttcaccacgccctccaaccagaacgtctaccaggtggactccctgcagtctacgtgaaaggcacccacctctcctagccgggagttgtccccattccttcagttcctcttgagcagcatcgaccaggctgctttctttctgtgtgtggccccgggtgtcaaaagtgtcaccagctgtctgtgttagacaaggttgccaagtgcaaaattggttattacggagggagagatttgctccattcattgtttttttggaaggacaggacatgctgtctctagatccagcaataggtttgcttctgtaccccagcctacccaagcagggactggacatccatccagatagagggtagcataggaatagggacaggagcatctgttctttaaggccttgtatggctgtttcctgttgcatctggaactaactatatatattgtcttcagtgaagactgattcaactttgggtatagtggagccaaagagatttttagctctgtatttgcggtatcggtttagaagacaaaaaaaattaaaacctgatacttttatctgattattgtaaatatttgatcttcaatcacttgacagtgtttgtttggcttgtatttgttttttatctttgggcttaaaagagatccaaagagagaaagagcagtatgccacttcttagaacaaaagtataaggaaaaaaatgttctttttaatccaaagggtatatttgcagcatgcttgaccttgatgtaccaattctgacggcattttcgtggatattattatcactaagactttgttatgatgaggtcttcagtctctttcatatatcttccttgtaactttttttttcctcttaatgtagttttgactctgccttacctttgtaaatatttggcttacagtgtctcaaggggtattttggaaagacaccaaaattgtgggttcacttttttttttttttttaaataacttcagctgtgctaaacagcatattacctctgtacaaaattcttcagggagtgtcacctcaaatgcaatactttgggttggtttctttccttttaaaaaaaaatacgaaactggaagtgtgtgtatgtgtgcgagtatgagcgcccatttggtggatgcaacaggttgagaggaagggagaattaacttgctccatgatgttcgtggtgtaaagttttgagctggaatttattataagaatgtaaaaccttaaattattaataaataactattttggctaaaaaaaaaaaaaaaaaaaaaaaaaaaaaaaagggcggcc

SEQ ID NO: 2 - protein, 381 aa, Mus musculus
MIDTLRPVPFASEMAICKTVSWLNEQLELGNERLLLMDCRPQELYESSHIESAINVAIPGIMLRRLQKGNLPVRALFTRCEDRDRFTRRCGTDTVVLYDENSSDWNENTGGESVLGLLLKKLKDEGCRAFYLEGGFSKFQAEFALHCETNLDGSCSSSSPPLPVLGLGGLRISSDSSSDIESDLDRDPNSATDSDGSPLSNSQPSFPVEILPFLYLGCAKDSTNLDVLEEFGIKYILNVTPNLPNLFENAGEFKYKQIPISDHWSQNLSQFFPEAISFIDEARGKNCGVLVHCLAGISRSVTVTVAYLMQKLNLSMNDAYDIVKMKKSNISPNFNFMGQLLDFERTLGLSSPCDNRVPTPQLYFTTPSNQNVYQVDSLQST

SEQ ID NO: 3 - DNA, 1143 bp, Mus musculus
  CDS (1)...(1143)
atgatagatacgctcagacccgtgcccttcgcgtcggaaatggcgatctgcaagacggtgtcgtggctcaacgagcagctggagctgggcaacgaacggcttctgctgatggactgccgaccacaggagctgtacgagtcgtcacacatcgaatctgccattaatgtggccatccccggcatcatgctgcggcgtctgcagaagggcaacctgcccgtgcgtgcgctcttcacgcgctgcgaggaccgggaccgctttaccaggcgctgcggcaccgacaccgtggtgctgtacgacgagaatagcagcgactggaatgagaacactggtggagagtcggtcctcgggctgctgctcaagaaactcaaagacgagggctgccgggcgttctacctggaaggtggcttcagtaagttccaggccgagttcgccctgcactgcgagaccaatctagacggctcgtgcagcagcagttccccgcctttgccagtgctggggctcgggggcctgcggatcagctcggactcttcctcggacattgagtctgaccttgaccgagaccccaatagtgcaacggactctgatggcagcccgctgtccaacagccagccttccttcccggtggagattttgcccttcctttacctgggctgtgccaaggactcgaccaacttggacgtgttggaagagtttggcatcaagtacatcttgaatgtcacccccaatttgcccaatctgtttgagaatgcgggcgagttcaaatacaagcaaattcctatctcggatcactggagccaaaacctgtcccagtttttccctgaggccatttctttcatagatgaagcccgaggcaaaaactgtggtgtcctggtgcattgcttggcaggtatcagccgctctgtcaccgtgacagtggcgtacctcatgcagaagctcaacctgtccatgaacgatgcttacgacattgttaagatgaagaagtccaacatctcccccaacttcaacttcatgggccagctgcttgacttcgaaaggaccctgggactgagcagcccttgtgacaaccgtgtccccactccgcagctgtacttcaccacgccctccaaccagaacgtctaccaggtggactccctgcagtctacg

SEQ ID NO: 4 - DNA, 2741 bp, Mus musculus
  CDS (254)...(1609)
gtcgacccacgcgtccgggattctgctgccaggaagcggccggcgcacgagtggcgcgaggcttggcttccacgtgcgagcctgctccaccaccccgccctctccgagcgcccaagcctgggggatccgtctccatgaacgatacctacgccctgccgtggcactagctgtggccaggacgtccgggtgcagtcgcctgcctctggatctcgctccagcacccactgcccgacgtgcaggacgctgcgcgaccatggagagtctgagtcggtcatgcctgtggctgcgtcaggagctgtcgtccccgcgcccacagcttctgctgctggactgccgcagccgggagctgtatgagtcggcgcgcatctgcggggcgctgagcgtggccctgccctcgctgatgctgcgccgcctgcggagggggagcatgtcggtgcggtcgctcttgcctgggccaccactgcagcctcccccaccggcccctgtgctcctgtacgaccagggtagcagtggccagtgccatcgtgaagcagcggaggctgaagcgaaggccgaacctaaggctgaagcggaggccgaacctaaggctgaagcggaggccgaacctaaggctgaagcggagaacgaagcggaggctaaagcggaggccaaagcggaggccaaagcggaggctaaagcggaggccaaagcggaggccaaagcggaggccaaagcggaggctaaagcggaggccaaagcggaggccaaagaagagtgggatgctgactcggtgctgggcgtcctgctccagaagctgcgggaagaaggctacccagcatactacctacagggtggcttcagcaagttccaggccgagtgtcctcacctgtgtgaaaccagcttcagtggtcgtgccggctcaggcctgggctcaatgtctagcccagtgcctgtggtggggctaggtggcctgtgcttgagctctgatttctctgatgcagagtctgaggctgaccgtgacaccttgagctgtggcctagattctgagaacaccacatcccctccagctgggctgctaccacccttcccagtccagatcctgcccaatctctacctaggcagtgctcgagattcagccaatttggagagcctggccaagcttggcatccgctatatcctcaatgtcacccccaaccttcctaacctctttgagaagaatggtgacttccactacaagcagatccccatctctgaccattggagccagaatctgtcccagttctttccggaggccattgcattcattgatgaggccttgtcgcagaactgtggggtgctcgtccactgtctggcaggggtcagccgctctgtcactgtcaccgtggcctacctcatgcagaagcttcacctctcactcaacgatgcctacgacttggtcaagcggaagaagtctaacatctcacctaacttcaactttatggggcagctgctagactttgaacgcagcctgcgcctgggggggaagcgctccggggggcggggcagtgggggaccagagtccaccgtctcagacccaccttccttctttactacccctaccagtgatggggtctttgagctggaccccacataaagccgcatggagctgataggctggtccctacttatgacccctagtataagcagggatgggatgcagccataagcagggagaggggaaaggggaaggggggagggaaagcgcaatacctcatgtggggatggtggagcaaggcaggaacagagtagaaacctgccccacccactcaaggccagccccagattctatttcacgtgcctactggtgggcccgggcttcgggcctgaccctcaccgctctagagcggggccatgctccagaacctgcctcgtctgtgactgttactttcttttttcagggtgggagtggggcttcctatccaagagaccattccagattgctgcctgggccctggcccttcttgctgtttcattttcaggaagagtgtatcattctttgccacaggaacctgctcttggctccatacctgtccccgaatgcccacctgtgggagcagacctggccactttgactcgtgtagactccttcctgcttctctcactagggcttagacttcttggaactgtagggtgtgaacccagagacacaatggctgctctccgcatccacagggacagcctctctgggtggctttttgctgggcagtgtcacatgggagggcatgggcctgggagccaatgggcgagatcccagccacacatctggtgccctgactgtttctttcctcctcccccagatgtcttgacaggatcactgagactctttgtgactgagggtggtcaaactgccaccggaggagatagggtctcagagctagagctgagggggagggagggaaagatgaaggcctcaattttgctactgtgagtctcacacagccactgttggtttggggggcagggaaggggccaagctgcagacacacatggtcattcattgcagtccacaccccgttgtagactctgtgtgtgtgtgtgtgtgtgtgtgtgtgtgtacacactagcccactcacatactcagcctggagctggcaatacctgtggcagcaggaccatcttgtctttgtttttgtaatccacatcataatcacattctttcattgttactcctgaataaacagtttatttaaggtactgaaaaaaaaaaaaaaaagggcggccgc

SEQ ID NO: 5 - protein, 452 aa, Mus musculus
MESLSRSCLWLRQELSSPRPQLLLLDCRSRELYESARICGALSVALPSLMLRRLRRGSMSVRSLLPGPPLQPPPPAPVLLYDQGSSGQCHREAAEAEAKAEPKAEAEAEPKAEAEAEPKAEAENEAEAKAEAKAEAKAEAKAEAKAEAKAEAKAEAKAEAKAEAKEEWDADSVLGVLLQKLREEGYPAYYLQGGFSKFQAECPHLCETSFSGRAGSGLGSMSSPVPVVGLGGLCLSSDFSDAESEADRDTLSCGLDSENTTSPPAGLLPPFPVQILPNLYLGSARDSANLESLAKLGIRYILNVTPNLPNLFEKNGDFHYKQIPISDHWSQNLSQFFPEAIAFIDEALSQNCGVLVHCLAGVSRSVTVTVAYLMQKLHLSLNDAYDLVKRKKSNISPNFNFMGQLLDFERSLRLGGKRSGGRGSGGPESTVSDPPSFFTTPTSDGVFELDPT

SEQ ID NO: 6 - DNA, 1356 bp, Mus musculus
  CDS (1)...(1356)
atggagagtctgagtcggtcatgcctgtggctgcgtcaggagctgtcgtccccgcgcccacagcttctgctgctggactgccgcagccgggagctgtatgagtcggcgcgcatctgcggggcgctgagcgtggccctgccctcgctgatgctgcgccgcctgcggagggggagcatgtcggtgcggtcgctcttgcctgggccaccactgcagcctcccccaccggcccctgtgctcctgtacgaccagggtagcagtggccagtgccatcgtgaagcagcggaggctgaagcgaaggccgaacctaaggctgaagcggaggccgaacctaaggctgaagcggaggccgaacctaaggctgaagcggagaacgaagcggaggctaaagcggaggccaaagcggaggccaaagcggaggctaaagcggaggccaaagcggaggccaaagcggaggccaaagcggaggctaaagcggaggccaaagcggaggccaaagaagagtgggatgctgactcggtgctgggcgtcctgctccagaagctgcgggaagaaggctacccagcatactacctacagggtggcttcagcaagttccaggccgagtgtcctcacctgtgtgaaaccagcttcagtggtcgtgccggctcaggcctgggctcaatgtctagcccagtgcctgtggtggggctaggtggcctgtgcttgagctctgatttctctgatgcagagtctgaggctgaccgtgacaccttgagctgtggcctagattctgagaacaccacatcccctccagctgggctgctaccacccttcccagtccagatcctgcccaatctctacctaggcagtgctcgagattcagccaatttggagagcctggccaagcttggcatccgctatatcctcaatgtcacccccaaccttcctaacctctttgagaagaatggtgacttccactacaagcagatccccatctctgaccattggagccagaatctgtcccagttctttccggaggccattgcattcattgatgaggccttgtcgcagaactgtggggtgctcgtccactgtctggcaggggtcagccgctctgtcactgtcaccgtggcctacctcatgcagaagcttcacctctcactcaacgatgcctacgacttggtcaagcggaagaagtctaacatctcacctaacttcaactttatggggcagctgctagactttgaacgcagcctgcgcctgggggggaagcgctccggggggcggggcagtgggggaccagagtccaccgtctcagacccaccttccttctttactacccctaccagtgatggggtctttgagctggaccccaca

(19) United States
(12) Patent Application Publication
(10) Pub. No.: US 2003/0022857 A1
(43) Pub. Date: Jan. 30, 2003

(54) Compositions and methods for the treatment of body weight disorders, including obesity

(75) Inventor: Haiyan Xu, Weymouth, MA (US)

Correspondence Address: MILLENNIUM PHARMACEUTICALS, INC., 75 Sidney Street, Cambridge, MA, 02139, US

(73) Assignee: Millennium Pharmaceuticals, Inc.

(21) Appl. No.: 10/184,832

(22) Filed: Jun. 28, 2002

Related U.S. Application Data
(60) Provisional application No. 60/303,250, filed on Jul. 5, 2001.

(51) Int. Cl. A61K 48/00; A61K 31/00; A61K 38/17; A61K 39/395

(52) U.S. Cl. 514/44; 514/12; 424/145.1; 514/1

(57) ABSTRACT

The invention provides isolated nucleic acids molecules, designated MKP nucleic acid molecules, which encode novel MAP kinase phosphatase family members, and their use in metabolic disorders. The invention also provides antisense nucleic acid molecules, recombinant expression vectors containing MKP nucleic acid molecules, host cells into which the expression vectors have been introduced, and nonhuman transgenic animals in which a MKP gene has been introduced or disrupted. The invention still further provides isolated MKP proteins, fusion proteins, antigenic peptides and anti-MKP antibodies. Diagnostic and therapeutic methods utilizing compositions of the invention are also provided.

### CROSS REFERENCE TO RELATED APPLICATIONS

This application claims the benefit of U.S. Provisional Application No. 60/303,250, filed Jul. 5, 2001 the contents of which are incorporated herein by this reference.

### BACKGROUND OF THE INVENTION

Body weight disorders, including eating and other disorders affecting regulation of body fat, represent major health problems in all industrialized countries. Obesity, the most prevalent of eating disorders, for example, is the most important nutritional disorder in the western world, with estimates of its prevalence ranging from 30% to 50% within the middle-aged population. Other body weight disorders, such as anorexia nervosa and bulimia nervosa, which together affect approximately 0.2% of the female population of the western world, also pose serious health threats. Further, such disorders as anorexia and cachexia (wasting) are also prominent features of other diseases such as cancer, cystic fibrosis, and AIDS.

Obesity, defined as an excess of body fat relative to lean body mass, also contributes to other diseases. For example, this disorder is responsible for increased incidence of diseases such as coronary artery disease, hypertension, stroke, diabetes, hyperlipidemia, and some cancers (See, e.g., Nishina, P. M. et al., 1994, Metab. 43: 554-558; Grundy, S. M. & Barnett, J. P., 1990, Dis. Mon. 36: 641-731). Obesity is not merely a behavioral problem, i.e., the result of voluntary hyperphagia. Rather, the differential body composition observed between obese and normal subjects results from differences in both metabolism and neurologic/metabolic interactions. These differences seem to be, to some extent, due to differences in gene expression, and/or level of gene products or activity. The nature, however, of the genetic factors which control body composition are unknown, and attempts to identify molecules involved in such control have generally been empiric, and the parameters of body composition and/or substrate flux have not yet been identified (Friedman, J. M. et al., 1991, Mammalian Gene 1:130-144).

The epidemiology of obesity strongly shows that the disorder exhibits inherited characteristics (Stunkard, 1990, N. Eng. J. Med. 322:1483). Moll et al., have reported that, in many populations, obesity seems to be controlled by a few genetic loci (Moll et al. 1991, Am. J. Hum. Gen. 49:1243). In addition, human twin studies strongly suggest a substantial genetic basis in the control of body weight, with estimates of heritability of 80-90% (Simopoulos, A. P. & Childs B., eds., 1989, in “Genetic Variation and Nutrition in Obesity”, World Review of Nutrition and Diabetes 63, S. Karger, Basel, Switzerland; Borjeson, M., 1976, Acta. Paediatr. Scand. 65:279-287).

In other studies, non-obese persons who deliberately attempted to gain weight by systematically over-eating were found to be more resistant to such weight gain and able to maintain an elevated weight only by very high caloric intake. In contrast, spontaneously obese individuals are able to maintain their status with normal or only moderately elevated caloric intake. In addition, it is a commonplace experience in animal husbandry that different strains of swine, cattle, etc., have different predispositions to obesity. Studies of the genetics of human obesity, and of animal models of obesity demonstrate that obesity results from complex defective regulation of both food intake, food induced energy expenditure, and of the balance between lipid and lean body anabolism.

There are a number of genetic diseases in man and other species which feature obesity among their more prominent symptoms, along with, frequently, dysmorphic features and mental retardation. For example, Prader-Willi syndrome (PWS; reviewed in Knoll, J. H. et al., 1993, Am. J. Med. Genet. 46: 2-6) affects approximately 1 in 20,000 live births, and involves poor neonatal muscle tone, facial and genital deformities, and, generally, obesity.

In addition to PWS, many other pleiotropic syndromes have been characterized which include obesity as a symptom. These syndromes are genetically straightforward, and appear to involve autosomal recessive alleles. Such diseases include, among others, Ahlstroem, Carpenter, Bardet-Biedl, Cohen, and Morgagni-Stewart-Monel Syndromes.

Animals having mutations which lead to syndromes that include obesity symptoms have also been identified. Researchers have used these animals to develop models to study obesity. The best studied animal models for genetic obesity are mice which contain the autosomal recessive mutations ob/ob (obese) and db/db (diabetes). These mutations are on chromosomes 6 and 4, respectively, but lead to clinically similar pictures of obesity, evident starting at about 1 month of age, which include hyperphagia, severe abnormalities in glucose and insulin metabolism, very poor thermoregulation and non-shivering thermogenesis, and extreme torpor and underdevelopment of the lean body mass. Restriction of the diet of these animals to restore a more normal body fat mass to lean body mass ratio is fatal.

Other animal models contain the autosomal recessive mutations tub/tub (tubby) or fa/fa (fatty), and bear similarity to the ob/ob and db/db mice. One difference is that, while fa/fa rats are very-sensitive to cold, their capacity for non-shivering thermogenesis is normal. Torpor seems to play a larger part in the maintenance of obesity in fa/fa rats than in the mice mutants. In addition, inbred mouse strains such as NZO mice and Japanese KK mice are moderately obese. Certain hybrid mice, such as the Wellesley mouse, become spontaneously fat. Further, several desert rodents, such as the spiny mouse, do not become obese in their natural habitats, but do become so when fed on standard laboratory feed.

Homozygous mutations at either the fat or tub loci cause obesity which develops more slowly than that observed in ob and db mice (Coleman, D. L., and Eicher, E. M., 1990, J. Heredity 81:424-427), with tub obesity developing slower than that observed in fat animals. This feature of the tub obese phenotype makes the development of tub obese phenotype closest in resemblance to the manner in which obesity develops in humans. Even so, however, the obese phenotype within such animals can be characterized as massive in that animals eventually attain body weights which are nearly two times the average weight seen in normal mice.

The fat mutation has been mapped to mouse chromosome 8, while the tub mutation has been mapped to mouse chromosome 7. According to Naggert et al., the fat mutation has recently been identified (Naggert, J. K., et al., 1995, Nature Genetics 10:135-141). Specifically, the fat mutation appears to be a mutation within the Cpe locus, which encodes the carboxypeptidase (Cpe) E protein. Cpe is an exopeptidase involved in the processing of prohormones, including proinsulin.

Attempts have been made to utilize such animal models in the study of molecular causes of obesity. For example, adipsin, a murine serine protease with activity closely similar to human complement factor D, produced by adipocytes, has been found to be suppressed in ob/ob, db/db and MSG-induced obesity (Flier, 1987, Science 237:405). The suppression of adipsin precedes the onset of obesity in each model (Lowell, 1990, Endocrinology 126:1514). Further studies have mapped the locus of the defect in these models to activity of the adipsin promoter (Platt, 1989, Proc. Natl. Acad. Sci. USA 86:7490). Further, alterations have been found in the expression of neurotransmitter peptides in the hypothalamus of the ob/ob mouse (Wilding, 1993, Endocrinology 132:1939), of glucose transporter proteins in islet β-cells (Ohneda, 1993, Diabetes 42:1065), and of the levels of G-proteins (McFarlane-Anderson, 1992, Biochem. J. 282:15).

In summary, obesity, which poses a major, worldwide health problem, represents a complex, highly heritable trait. To date, no gene, in humans, has been found which is causative in the processes leading to obesity. Given the importance of understanding body weight homeostasis and, further, given the severity and prevalence of disorders, including obesity, which affect body weight and body composition, there exists a great need for the systematic identification of genes involved in these processes and disorders.

### SUMMARY OF THE INVENTION

The invention is based, at least in part, on the discovery of novel genes encoding MAP kinase phosphatase protein family members (i.e., MKP family members), which are enzymes implicated in signal transduction pathways (e.g., the insulin signaling pathway), and which have also been shown to be differentially regulated in metabolic-related tissues (e.g., insulin-responsive tissues). The nucleotide sequences of cDNAs encoding MKP3 and MKP4 are shown in SEQ ID NO:1 and SEQ ID NO:4, and the amino acid sequences of MKP3 and MKP4 polypeptides are shown in SEQ ID NO:2 and SEQ ID NO:5. In addition, the nucleotide sequences of the coding regions are depicted in SEQ ID NO:3 and SEQ ID NO:6.

Accordingly, in one aspect, the invention features nucleic acid molecules that encode MKP3 or MKP4 proteins or polypeptides, e.g., a biologically active portion of the MKP3 or MKP4 protein. In a preferred embodiment the isolated nucleic acid molecules encode polypeptides having the amino acid sequences SEQ ID NO:2 and SEQ ID NO:5. In other embodiments, the invention provides isolated MKP3 and MKP4 nucleic acid molecules having the nucleotide sequences of one of SEQ ID NO:1, SEQ ID NO:3, SEQ ID NO:4, and SEQ ID NO:6.

In still other embodiments, the invention provides nucleic acid molecules that have sequences that are substantially identical (e.g., naturally occurring allelic variants) to the nucleotide sequences of one of SEQ ID NO:1, SEQ ID NO:3, SEQ ID NO:4, and SEQ ID NO:6. In other embodiments, the invention provides nucleic acid molecules which hybridize under stringent hybridization conditions with a nucleic acid molecule having a sequence comprising the nucleotide sequence of one of SEQ ID NO:1, SEQ ID NO:3, SEQ ID NO:4, and SEQ ID NO:6, wherein the nucleic acids encode full length MKP3 and MKP4 proteins or an active fragment thereof.

In a related aspect, the invention further provides nucleic acid constructs that include MKP3 and MKP4 nucleic acid molecules described herein. In certain embodiments, the nucleic acid molecules of the invention are operatively linked to native or heterologous regulatory sequences. Also included are vectors and host cells containing the MKP3 and MKP4 nucleic acid molecules of the invention, e.g., vectors and host cells suitable for producing MKP3 and MKP4 polypeptides.

In another related aspect, the invention provides nucleic acid fragments suitable as primers or hybridization probes for detection of MKP3 and MKP4-encoding nucleic acids.

In still another related aspect, isolated nucleic acid molecules that are antisense to MKP3 and MKP4-encoding nucleic acid molecules are provided.

In another aspect, the invention features MKP3 and MKP4 polypeptides, and biologically active or antigenic fragments thereof, that are useful, e.g., as reagents or targets in assays applicable to treatment and diagnosis of MKP3 and MKP4-mediated or MKP3 and MKP4-related disorders.

In another embodiment, the invention provides MKP3 and MKP4 polypeptides having a MKP3 and MKP4 activity. Preferred polypeptides are MKP3 and MKP4 proteins including at least one conserved MKP3 and MKP4 domain, e.g., an MKP family member domain (e.g., a dual specificity phosphatase, catalytic domain); and MKP3 and MKP4 proteins including one or more transmembrane domains.

In other embodiments, the invention provides MKP3 and MKP4 polypeptides, e.g., MKP3 and MKP4 polypeptides having the amino acid sequences shown in SEQ ID NO:2 and SEQ ID NO:5; amino acid sequences that are substantially identical to the amino acid sequences shown in SEQ ID NO:2 and SEQ ID NO:5; or amino acid sequences encoded by nucleic acid molecules having nucleotide sequences which hybridize under stringent hybridization conditions to a nucleic acid molecule comprising the nucleotide sequence of any of SEQ ID NO:1, SEQ ID NO:3, SEQ ID NO:4, and SEQ ID NO:6, wherein the nucleic acids encode full length MKP3 and MKP4 proteins or an active fragment thereof.

In a related aspect, the invention further provides nucleic acid constructs that include MKP3 and MKP4 nucleic acid molecules described herein.

In a related aspect, the invention provides MKP3 and MKP4 polypeptides or fragments operatively linked to non-MKP3 and MKP4 polypeptides to form fusion proteins.

In another aspect, the invention features antibodies and antigen-binding fragments thereof, that react with, or more preferably, specifically or selectively bind, MKP3 and MKP4 polypeptides.

In another aspect, the invention provides methods of screening for compounds that modulate the expression or activity of the MKP3 and MKP4 polypeptides or nucleic acids.

In still another aspect, the invention provides a process for modulating MKP3 and MKP4 polypeptide or nucleic acid expression or activity, e.g., using the compounds identified in the screens described herein. In certain embodiments, the methods involve treatment of conditions related to aberrant activity or expression of the MKP3 and MKP4 polypeptides or nucleic acids, such as metabolic and body weight disorders, (e.g., obesity, diabetes, anorexia nervosa, bulimia nervosa, and cachexia). The invention also relates to methods of using the reagents, compositions, and compounds described herein to treat and/or prevent metabolic and body weight disorders described herein.

The invention further provides methods for the identification of compounds which modulate the expression of genes or the activity of gene products involved in body weight disorders and processes relevant to appetite and/or body weight regulation. Still further, the invention describes methods for the treatment of body weight disorders and the modulation of metabolic processes in mammals which may involve the administration of such compounds to individuals exhibiting body weight disorder symptoms or tendencies or in need of regulation of aberrant metabolism.

Additionally, the invention describes methods for prognostic and diagnostic evaluation of various body weight disorders, and for the identification of subjects exhibiting a predisposition to such disorders.

The invention also provides assays for determining the activity of or the presence or absence of MKP3 and MKP4 polypeptides or nucleic acid molecules in a biological sample, including for disease diagnosis.

In further aspect the invention provides assays for determining the presence or absence of a genetic alteration in a MKP3 and MKP4 polypeptide or nucleic acid molecule, including for disease diagnosis.

Other features and advantages of the invention will be apparent from the following detailed description, and from the claims.

### DESCRIPTION OF THE FIGURE

FIG. 1 shows that the differentiation of preadipocytes (i.e., into mature fat cells (adipocytes)) can be inhibited by the ectopic expression of MKP4 (the details of which are described herein). FIG. 1 shows adipocytes stained for lipid content, which indicates whether or not they have differentiated from preadipocytes. The adipocytes were infected with retrovirus containing mouse MKP4 DNA (and adipocytes in which just the retroviral vector (control) was transfected), and with retrovirus containing mouse MKP3 DNA (and control vector)). Northern blots were run to corroborate the stable expression of mouse MKP3 and mouse MKP4 that were used in the experiments depicted in FIG. 1. The different lanes in the Northern blots represent mouse MKP4 and mouse MKP3 expression in the absence and presence of mitogen-containing serum, which induced MKP upregulation when encountered.

### DETAILED DESCRIPTION OF THE INVENTION

Methods and compositions are described herein for the modulation of body weight-related processes, including, for example, treatment of metabolic and body weight disorders such as obesity, diabetes, and cachexia, and modulation of body weight and thermogenesis. Genes are described which are differentially expressed in body weight disorder states, relative to their expression in normal, or non-body weight disorder states, and/or which are differentially expressed in response to manipulations relevant to appetite and/or body weight regulation. Additionally, genes are described whose gene products exhibit an ability to interact with gene products involved in body weight disorders and/or with gene products which are relevant to appetite and/or body weight regulation. Methods for the identification of such genes are also described, as are the gene products of such genes.

“Differential expression” as used herein refers to both quantitative as well as qualitative differences in the genes' temporal and/or tissue expression patterns. Thus, a differentially expressed gene may qualitatively have its expression activated or completely inactivated in normal versus body weight disorder states, or under control versus experimental conditions. Such a qualitatively regulated gene will exhibit an expression pattern within a given tissue or cell type which is detectable in either control or body weight disorder subjects, but is not detectable in both. Alternatively, such a qualitatively regulated gene will exhibit an expression pattern within a given tissue or cell type which is detectable in either control or experimental subjects, but is not detectable in both. “Detectable”, as used herein, refers to an RNA expression pattern which is detectable via the standard techniques of differential display, RT-PCR and/or Northern analyses, which are well known to those of skill in the art.

Alternatively, a differentially expressed gene may have its expression modulated, i.e., quantitatively increased or decreased, in normal versus body weight disorder states, or under control versus experimental conditions. The degree to which expression differs in normal versus body weight disorder or control versus experimental states need only be large enough to be visualized via standard characterization techniques, such as, for example, the differential display technique described below. Other such standard characterization techniques by which expression differences may be visualized include, but are not limited to, quantitative RT (reverse transcriptase) PCR and Northern analyses.

Described herein are methods for the identification of compounds which modulate the expression of genes or the activity of gene products involved in body weight disorders and processes relevant to appetite and/or body weight regulation. Additionally, described below are methods for the treatment of body weight disorders. Also discussed below are methods for prognostic and diagnostic evaluation of various body weight disorders, and for the identification of subjects exhibiting a predisposition to such disorders.

Among the genes and gene products of the invention are members of the MAP (mitogen-activated protein) kinase phosphatase family (herein used interchangeably with “MKP family”), including but not limited to, mouse MKP3 and mouse MKP4. As demonstrated in the Examples presented herein, MKP family members are expressed in tissues (e.g., muscle and adipose tissue) involved in such metabolic processes as thermogenesis, and are upregulated in mice models of obesity (i.e., as compared to the wild types).

The term “family” when referring to the protein and nucleic acid molecules of the invention means two or more proteins or nucleic acid molecules having a common structural domain or motif and having sufficient amino acid or nucleotide sequence homology as defined herein. Such family members can be naturally or non-naturally occurring and can be from either the same or different species. For example, a family can contain a first protein of human origin as well as other distinct proteins of human origin, or alternatively, can contain homologs of non-human origin, e.g., rat or mouse proteins. Members of a family also can have common functional characteristics.

MKP family members are enzymes implicated in signal transduction pathways, e.g., pathways in which extracellular signals (e.g., signals for growth and differentiation) are transmitted to the cell nucleus. MAP kinases (MAPKs), which are substrates on which MKP family members act, are key enzymes in a variety of signal transduction pathways (e.g., the insulin signaling pathway), and are responsible for the inward passage of messages received at the cell surface by growth factor and hormone receptors. MAPKs have been demonstrated to phosphorylate and regulate numerous cellular proteins, including growth factor receptors, transcription factors, cytoskeletal proteins, phospholipase, and other protein kinases. MAPKs have been observed during growth factor stimulation of DNA synthesis, and are involved in such processes as differentiation, secretion, and stimulation of glycogen synthesis.

MAP kinases comprise such classes as extracellular signal-related kinases (ERKs), which mediate neuronal differentiation and growth factor and oncogenic transformation; c-Jun N-terminal kinases/stress-activated protein kinases (JNKs/SAPKs), which are involved in mediating platelet aggregation and secretion, generating inflammatory cytokines, and in pathways leading to apoptotic cell death; and p38/RK/CSBP kinases, which play roles similar to those of JNKs/SAPKs and have been shown to block the production and action of inflammatory cytokines such as tumor necrosis factor (TNF) and interleukin-1 (IL-1)(Kumar S. et al. (2001) J Cell Physiol 187(3):294-303).

MAP kinases are activated by kinases called “MAP kinase kinases” (also known as “MEKs”), and are deactivated by MAP kinase phosphatase family members. Both MEKs and MKPs are known as dual-specificity enzymes because they exert their effects (phosphorylation and dephosphorylation, respectively) on serine or threonine, as well as on tyrosine, residues within their substrates (MAPKs, for instance). The interplay between MEKs and MKPs is critical to modulate signal transduction, and to, in turn, modulate such processes as cellular growth and differentiation.

MAP kinases are key players in the insulin signaling pathway, of which the Ras-Raf-MAPK cascade (a central effector of cellular differentiation in development) is a component. Insulin is known to have an inhibitory effect on hepatic gene expression and specifically on the phosphoenolpuryvate carboxykinase (PEPCK) gene, a key enzyme in glucogenesis of the liver. Experiments were performed in which MKP family members (e.g., mouse MKP3 and mouse MKP4) were discovered to reverse insulin's antagonistic effects, e.g., MKP family members could induce transcription of PEPCK that was stopped by insulin. Specifically, expression vectors were constructed with PEPCK promoters links to alkaline phosphatase. While insulin repressed the promoter, and hence the expression of the constructs, the presence of MKP family members (e.g., mouse MKP3 and mouse MKP4) could induce its expression in rat hepatoma cells.

Although the insulin signaling pathway is known not to be implicated in insulin's antagonism of PEPCK expression, the experiments nonetheless led to an interest in determining how MKP family members could reverse insulin's effects on the metabolic arm. Therefore, the experiments led to an interest in testing the effects of MKP family members in such insulin-related systems as mice models of obesity and insulin-resistance, and an interest in MKP family members' role in metabolic disorders in general.

In a preferred embodiment, the genes of the invention are mammalian MKP3s. MKP3 (also known as pyst1) differs from other MKP family members in at least the following ways: it seems to strongly prefer ERKs specifically (whereas MKP1 and MKP2 show relatively equal catalytic activity towards MAPK classes); it is not induced by such stimuli as heat shock, sodium arsenic, and osmotic shock; it is not an intermediate early gene; and it is located in the cytosol, rather than the nucleus. Human MKP3 has GenBank Accession number X93920, and is described at least in Groom et al. (1996) EMBO Journal 15(14):3621-3632).

In another preferred embodiment, the differentially expressed genes (in body weight disorder states) are mammalian MKP4s. MPK4 displays selectivity for the ERK class of MAP kinases, and is able to catalyze substrates in both the cell nucleus and cytosol. MPK is thought to play a role as a tumor suppressor, due to its ability to inhibit MAP kinases which are activated by oncogenes. Human MKP4 has GenBank Accession number Y08302, and is described at least in Muda et al. (1997) JBC 272(8):5141-5151.

Mouse MKP3 and MKP4 are still other preferred embodiments of differentially expressed genes in body weight disorder states. The mouse MKP3 sequence (SEQ ID NO:1), which is approximately 2700 nucleotides long including untranslated regions, contains a predicted methionine-initiated coding sequence of about 1143 nucleotides (SEQ ID NO:3), not including the termination codon. The coding sequence encodes a 381 amino acid protein (SEQ ID NO:2), which is 97.9% identical to the human protein described in Groom et al., supra, as determined by an alignment performed using the GAP alignment program with a BLOSUM62 scoring matrix, and gap and length weight penalties of 12 and 4, respectively.

The mouse MKP4 sequence (SEQ ID NO:4), which is approximately 2741 nucleotides long including untranslated regions, contains a predicted methionine-initiated coding sequence of about 1356 nucleotides (SEQ ID NO:6), not including the termination codon. The coding sequence encodes a 452 amino acid protein (SEQ ID NO:5), which is 71.9% identical to the human protein described in Muda et al., supra, as determined by an alignment performed using the GAP alignment program with a BLOSUM62 scoring matrix, and gap and length weight penalties of 12 and 4, respectively.

In one embodiment, MKP family members of the invention (e.g., mouse MKP3 and mouse MKP4) can include at least one dual specificity phosphatase, catalytic domain (Prosite PF00782). Furthermore, MKP family members can include at least one or more, preferably two or more, transmembrane domains.

To identify the presence of domains in a MKP protein sequences, and to make the determination that a polypeptide or protein of interest has a particular profile, the amino acid sequence of the protein can be searched against the Pfam database of Hidden Markov Models (HMMs) (e.g., the Pfam database, release 2.1) using the default parameters (http://www.sanger.ac.uk/Software/Pfam/HMM_search). For example, the hmmsf program, which is available as part of the HMMER package of search programs, is a family specific default program for MILPAT0063 and a score of 15 is the default threshold score for determining a hit. Alternatively, the threshold score for determining a hit can be lowered (e.g., to 8 bits). A description of the Pfam database can be found in Sonhammer et al. (1997) Proteins 28:405-420 and a detailed description of HMMs can be found, for example, in Gribskov et al. (1990) Meth. Enzymol. 183:146-159; Gribskov et al. (1987) Proc. Natl. Acad. Sci. USA 84:4355-4358; Krogh et al. (1994) J. Mol. Biol. 235:1501-1531; and Stultz et al. (1993) Protein Sci. 2:305-314, the contents of which are incorporated herein by reference.

As used herein, a “dual specificity phosphatase, catalytic domain” includes an amino acid sequence of about 120 to 160 amino acid residues in length and having a bit score for the alignment of the sequence to the immunoglobulin domain (HMM) of at least 240. As described infra, the dual specificity nature of some enzymes (e.g., MKPs) allows them to exert their effects on serine or threonine, as well as on tyrosine, residues within their substrates (e.g., to phosphorylate said residues). The dual specificity phosphatase, catalytic domain is found in such proteins, many of which are important in the control of cell growth, proliferation, differentiation and transformation.

In a preferred embodiment, the MKP family members of the invention possess dual specificity phosphatase, catalytic domains, or a region which includes at least 120 to 160 amino acids, preferably about 130 to 150 amino acids, more preferably about 135 to 145 amino acids, and has at least about 60%, 70% 80% 90% 95%, 99%, or 100% homology with a “dual specificity phosphatase, catalytic domain”, e.g., the dual specificity phosphatase, catalytic domain of mouse MKP3 or mouse MKP4 (e.g., residues 206 to 346 of SEQ ID NO:2 and residues 271 to 411 of SEQ ID NO:5, respectively).

In a preferred embodiment, the MKP family members of the invention (e.g., mouse MKP3 and mouse MKP4) have at least one or more, preferably two or more, “transmembrane domains” or regions which includes at least about 12 to 35, more preferably about 14 to 30, or 15 to 25 amino acid residues and has at least about 60%, 70% 80% 90% 95%, 99%, or 100% homology with a “transmembrane domain,” e.g., the transmembrane domains of the MKP family members of the invention (e.g., mouse MKP3 and mouse MKP4)(e.g., residues 153-170 and 289-308 of SEQ ID NO:2, and residues 214-237 and 354-373 of SEQ ID NO:5, respectively).

To identify the presence of a “transmembrane” domain in the sequence of a the MKP family member of the invention (e.g., in the mouse MKP3 and mouse MKP4 protein sequences), and to make the determination that a polypeptide or protein of interest has a particular profile, the amino acid sequence of the protein can be analyzed by a transmembrane prediction method that predicts the secondary structure and topology of integral membrane proteins based on the recognition of topological models (MEMSAT, Jones et al., (1994) Biochemistry 33:3038-3049).

MKP family members(e.g., mouse MKP3 and mouse MKP4) are expressed in a variety of tissues, predominantly in insulin-related (e.g., insulin-responsive) tissues in mice models of obesity (i.e., compared to in wild type mice). Although it has been known in the art that MKP family members are expressed in such tissues as kidney and placenta, metabolic-related tissues (e.g., adipose tissue) had not been examined in the art. As MKP4 was identified in a white adipose tissue (WAT) library during expression cloning, Northern blots were run in order to examine the tissue distribution of mouse MKP3 and mouse MKP4. The entire coding region of each gene was used as a probe (digested with BamHI and XbaI for MKP4, NotI/SalI for MKP3) in all of the Northern blots described herein.

As described in the Examples section infra, MKP family members are differentially expressed (i.e., upregulated) in predominantly insulin-related tissues in mice models of obesity. Furthermore, MKP family members (e.g., mouse MKP4) are upregulated in preadipocytes as they differentiate. However, overexpression of MKP family members (e.g., mouse MKP4) can inhibit preadipocyte differentiation (i.e., prevent them from becoming mature fat cells (adipocytes)).

As the MKP family member polypeptides of the invention (e.g., the mouse MKP3 and mouse MKP4 polypeptides) are upregulated in metabolic-related tissues (e.g., insulin-responsive), as they are upregulated during preadipocyte differentiation, as they can block the action of insulin (e.g., on the PEPCK promoter), and as mouse MKP4 overexpression can prevent preadipocyte differentiation, the MKP family members of the invention can be useful for developing novel diagnostic and therapeutic agents for metabolic-associated disorders, as described below.

As used herein, a “MKP activity”, “biological activity of MKP” or “functional activity of MKP”, refers to an activity exerted by a MKP family member protein, polypeptide or nucleic acid molecule (e.g., a mouse MKP3 and/or a mouse MKP4 protein, polypeptide, or nucleic acid molecule) on e.g., a MKP3 and MKP4-responsive cell or on a MKP3 and MKP4 substrate, e.g., a preadipocyte, or a MAP kinase, as determined in vivo or in vitro. In one embodiment, a MKP activity is a direct activity, such as an association with a MKP target molecule. A “target molecule” or “binding partner” is a molecule with which a MKP3 and MKP4 protein binds or interacts in nature.

As the MKP polypeptides of the invention can modulate MKP-mediated activities, they can be useful for developing novel diagnostic and therapeutic agents for metabolic or other MKP-associated disorders, as described below.

In an exemplary embodiment, MKP family members are enzymes for a MAP kinase substrate, e.g., a MAP kinase in the insulin pathway, e.g., a MAP kinase in the insulin pathway involved in a metabolic disorder (e.g., diabetes).

A MKP activity can also be an indirect activity, e.g., a cellular signaling activity mediated by interaction of a MKP protein with, for example, growth factor receptors and/or transcription factors. Based on the above-described sequence structures and similarities to molecules of known function, the MKP molecules of the invention can have similar biological activities as MAP kinase phosphatase family members. For example, the MKP proteins of the invention can have one or more of the following activities:. (1) the ability to modulate (e.g., inactivate, e.g., via dephosphorylation) enzymes involved in signaling pathways, e.g., MAP kinases; (2) the ability to reverse the effects of the activities of enzymes (e.g., MEKs) involved in signaling pathways; (3) the ability to modulate (e.g., inhibit) pathways via the modulation (e.g., inhibition) of components of said pathways (e.g., signal transduction pathways (e.g., those involved in cellular growth, mitogenesis, and differentiation)); (4) the ability to monitor, treat and/or diagnose disorders in which signaling pathways which MKP molecules of the invention are capable of modulating are involved (e.g., metabolic disorders, in which the insulin signaling pathways, e.g., is involved); (5) the ability to modulate (e.g., prevent) cellular differentiation (e.g., the differentiation of preadipocytes into adipocytes); (6) the ability to modulate glycogen synthesis, e.g., the differentiation or stimulation thereof; and (7) the ability to modulate cellular utilization of energy sources, e.g., cellular uptake of glucose.

Thus, the MKP molecules of the invention (e.g., mouse MKP3 and mouse MKP4) can act as novel diagnostic targets and therapeutic agents for controlling one or more disorders associated with MAP kinase phosphatases. Furthermore, due to the expression data described herein, the MKP molecules of the invention (e.g., mouse MKP3 and mouse MKP4) can act as novel diagnostic targets and therapeutic agents for controlling one or more metabolic disorders.

Examples of metabolic disorders which the MKP proteins, polypeptides, and nucleic acids of the invention can be used to monitor, treat and/or diagnose, include obesity (and appetite related disorders such as anorexia nervosa, bulimia nervosa, binge eating disorders, starvation, and cachexia) and disorders which arise as complications or byproducts of obesity, including but not limited to, cardiovascular disorders. As used herein, disorders involving the heart, or “cardiovascular disease” or a “cardiovascular disorder” includes a disease or disorder which affects the cardiovascular system, e.g., the heart, the blood vessels, and/or the blood. A cardiovascular disorder can be caused by an imbalance in arterial pressure, a malfunction of the heart, or an occlusion of a blood vessel, e.g., by a thrombus. A cardiovascular disorder includes, but is not limited to disorders such as arteriosclerosis, atherosclerosis, cardiac hypertrophy, ischemia reperfusion injury, restenosis, arterial inflammation, vascular wall remodeling, ventricular remodeling, rapid ventricular pacing, coronary microembolism, tachycardia, bradycardia, pressure overload, aortic bending, coronary artery ligation, vascular heart disease, valvular disease, including but not limited to, valvular degeneration caused by calcification, rheumatic heart disease, endocarditis, or complications of artificial valves; atrial fibrillation, long-QT syndrome, congestive heart failure, sinus node dysfunction, angina, heart failure, hypertension, atrial fibrillation, atrial flutter, pericardial disease, including but not limited to, pericardial effusion and pericarditis; cardiomyopathies, e.g., dilated cardiomyopathy or idiopathic cardiomyopathy, myocardial infarction, coronary artery disease, coronary artery spasm, ischemic disease, arrhythmia, sudden cardiac death, and cardiovascular developmental disorders (e.g., arteriovenous malformations, arteriovenous fistulae, raynaud's syndrome, neurogenic thoracic outlet syndrome, causalgia/reflex sympathetic dystrophy, hemangioma, aneurysm, cavernous angioma, aortic valve stenosis, atrial septal defects, atrioventricular canal, coarctation of the aorta, Ebstein's anomaly, hypoplastic left heart syndrome, interruption of the aortic arch, mitral valve prolapse, ductus arteriosus, patent foramen ovale, partial anomalous pulmonary venous return, pulmonary atresia with ventricular septal defect, pulmonary atresia without ventricular septal defect, persistance of the fetal circulation, pulmonary valve stenosis, single ventricle, total anomalous pulmonary venous return, transposition of the great vessels, tricuspid atresia, truncus arteriosus, ventricular septal defects). A cardiovascular disease or disorder also can include an endothelial cell disorder.

Further examples of metabolic disorders, which the MKP proteins, polypeptides, and nucleic acids of the invention can be used to monitor, treat and/or diagnose include lipid disorders (e.g., familial hypercholesteroliemia, polygenic hypercholesteroliemia, familial hypertriglyceridemia, familial lipoprotein lipase deficiency, combined hyperlipidemia, dysbetalipoproteinemia, sitosterolemia, Tangier disease, hypobetalipoproteinemia, lecithin:cholesterol acyltransferase (LCAT) deficiency, and cerebrotendinous xanthomatosis); diabetes (e.g., diabetes insipidus, diabetes mellitus (type I), diabetes mellitus (type II)); toxic and acquired metabolic diseases, including vitamin deficiencies such as thiamine (vitamin B₁) deficiency, vitamin B₁₂ deficiency, vitamin D deficiency, nephrotic syndrome, and vitamin-D related hypercalcemia; neurologic sequelae of metabolic disturbances, including hypoglycemia, hyperglycemia, and hepatic encephatopathy; acid maltase deficiency; galactosemia; maple syrup urine disease (MSUD); Niemann-Pick Disease; urea cycle disorders; gout and purine metabolic disorders (e.g., hyperuriceria, hypouricemia); lysosomal storage diseases (e.g., Fabry's disease, Gaucher's disease, fucosidosis, Krabbe's disease, Farber's disease); and glycogen storage diseases (e.g., liver glycogenoses, muscle glycogenoses).

Further examples of metabolic disorders include galactosemia, galactokinase deficiency, and other carbohydrate metabolic disorders; adipose tissue disorders (e.g., lipodystrophies, multiple symmetric lipomatosis, mediastinoabdominal lipomatosis, acute panniculitis (nodular fat necrosis), disseminated fat necrosis, and adiposis dolorosa (Dercum's disease)).

Still further examples of metabolic disorders include endocrine disorders; e.g., mucocutaneous candidiasis; hypoparathyroidism; adrenal insufficiency; hypogonadism; alopecia; hypothyroidism; malabsorption; chronic active hepatitis; vitiligo; pernicious anemia; adrenal insufficiency; Graves' disease; myasthenia gravis; celiac disease; ataxiatelangiectasia; psuedohypoparathyroidism; myotonic dystrophy; Noonan syndrome; Fanconi syndrome; Werner syndrome; hemochromatosis; hepatic porphyrias (e.g., acute intermittent porphyria (AIP)); and erythropoietic porphyrias (e.g., congenital erythropoietic porphyria (EPP)).

Furthermore, MKP proteins, polypeptides, and nucleic acids of the invention can be used to monitor, treat and/or diagnose disorders related to the regulation of body temperature (and thermogenesis, or generation of heat in an organism). Such disorders include fever, hyperthermia (e.g., drug induced hyperthermia, malignant hyperthermia), hypothermia, and heat stroke.

Furthermore, MKP proteins, polypeptides, and nucleic acids of the invention can be used to monitor, treat and/or diagnose disorders related to MAP kinase phosphatase (MKP) family members in general. For instance, dephosphorylation of MAP kinases and MAP kinase-like proteins by MKPs (e.g., mouse MKP3 and mouse MKP4) implicates MKP family members in the regulation of such processes as cellular mitogenesis and differentiation (Mourey et al., supra). Such cellular differentiative and mitogenic (e.g., proliferative) disorders which the MKP proteins, polypeptides, and nucleic acids of the invention can be used to treat include, but are not limited to, cancer, e.g., carcinoma, sarcoma, metastatic disorders or hematopoietic neoplastic disorders, e.g., leukemias. A metastatic tumor can arise from a multitude of primary tumor types, including but not limited to those of prostate, colon, lung, breast and liver origin.

As used herein, the term “cancer” (also used interchangeably with the terms, “hyperproliferative” and “neoplastic”) refers to cells having the capacity for autonomous growth, i.e., an abnormal state or condition characterized by rapidly proliferating cell growth. Cancerous disease states may be categorized as pathologic, i.e., characterizing or constituting a disease state, e.g., malignant tumor growth, or may be categorized as non-pathologic, i.e., a deviation from normal but not associated with a disease state, e.g., cell proliferation associated with wound repair. The term is meant to include all types of cancerous growths or oncogenic processes, metastatic tissues or malignantly transformed cells, tissues, or organs, irrespective of histopathologic type or stage of invasiveness. The term “cancer” includes malignancies of the various organ systems, such as those affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term “carcinoma” also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

Other proliferative disorders which the MKP family members of the invention (e.g., mouse MKP3 and mouse MKP4) can be used to monitor, treat and/or diagnose include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. Preferably, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML) (reviewed in Vaickus (1991) Crit Rev. in Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease.

The MKP protein, fragments thereof, and derivatives and other variants of the sequence in SEQ ID NO:2 and SEQ ID NO:5 thereof are collectively referred to as “polypeptides or proteins of the invention” or “MKP polypeptides or proteins”. Nucleic acid molecules encoding such polypeptides or proteins are collectively referred to as “nucleic acids of the invention” or “MKP nucleic acids.”

As used herein, the term “nucleic acid molecule” includes DNA molecules (e.g., a cDNA or genomic DNA) and RNA molecules (e.g., an mRNA) and analogs of the DNA or RNA generated, e.g., by the use of nucleotide analogs. The nucleic acid molecule can be single-stranded or double-stranded, but preferably is double-stranded DNA.

The term “isolated or purified nucleic acid molecule” includes nucleic acid molecules which are separated from other nucleic acid molecules which are present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and/or 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb or 0.1 kb of 5′ and/or 3′ nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or substantially free of chemical precursors or other chemicals when chemically synthesized.

As used herein, the term “hybridizes under stringent conditions” describes conditions for hybridization and washing. Stringent conditions are known to those skilled in the art and can be found in available references (e.g., Current Protocols in Molecular Biology, John Wiley & Sons, N.Y., 1989, 6.3.1-6.3.6). Aqueous and non-aqueous methods are described in that reference and either can be used. A preferred example of stringent hybridization conditions are hybridization in 6×sodium chloride/sodium citrate (SSC) at about 45° C., followed by one or more washes in 0.2×SSC, 0.1% (w/v) SDS at 50° C. Another example of stringent hybridization conditions are hybridization in 6×SSC at about 45° C., followed by one or more washes in 0.2×SSC, 0.1% (w/v) SDS at 55° C. A further example of stringent hybridization conditions are hybridization in 6×SSC at about 45° C., followed by one or more washes in 0.2×SSC, 0.1% (w/v) SDS at 60° C. Preferably, stringent hybridization conditions are hybridization in 6×SSC at about 45° C., followed by one or more washes in 0.2×SSC, 0.1% (w/v) SDS at 65° C. Particularly preferred stringency conditions (and the conditions that should be used if the practitioner is uncertain about what conditions should be applied to determine if a molecule is within a hybridization limitation of the invention) are 0.5 molar sodium phosphate, 7% (w/v) SDS at 65° C, followed by one or more washes at 0.2×SSC, 1% (w/v) SDS at 65° C.

As used herein, a “naturally-occurring” nucleic acid molecule refers to an RNA or DNA molecule having a nucleotide sequence that occurs in nature (e.g., encodes a natural protein).

As used herein, the terms “gene” and “recombinant gene” refer to nucleic acid molecules which include an open reading frame encoding a MKP protein, preferably a mammalian MKP protein, and can further include non-coding regulatory sequences, and introns.

An “isolated” or “purified” polypeptide or protein is substantially free of cellular material or other contaminating proteins from the cell or tissue source from which the protein is derived, or substantially free from chemical precursors or other chemicals when chemically synthesized. In one embodiment, the language “substantially free” means preparation of MKP protein having less than about 30%, 20%, 10% and more preferably 5% (by dry weight), of non-MKP protein (also referred to herein as a “contaminating protein”), or of chemical precursors or non-MKP chemicals. When the MKP protein or biologically active portion thereof is recombinantly produced, it is also preferably substantially free of culture medium, i.e., culture medium represents less than about 20%, more preferably less than about 10%, and most preferably less than about 5% of the volume of the protein preparation. The invention includes isolated or purified preparations of at least 0.01, 0.1, 1.0, and 10 milligrams in dry weight.

A “non-essential” amino acid residue is a residue that can be altered from the wild-type sequence of MKP (e.g., the sequence of SEQ ID NO:1 or SEQ ID NO:4) without abolishing or more preferably, without substantially altering a biological activity, whereas an “essential” amino acid residue results in such a change. For example, amino acid residues that are conserved among the polypeptides of the invention, e.g., those present in the dual specificity phosphatase, catalytic domain, are predicted to be particularly unamenable to alteration.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a MKP protein is preferably replaced with another amino acid residue from the same side chain family. Alternatively, in another embodiment, mutations can be introduced randomly along all or part of a MKP coding sequence, such as by saturation mutagenesis, and the resultant mutants can be screened for MKP biological activity to identify mutants that retain activity. Following mutagenesis of SEQ ID NO:1 or SEQ ID NO:4, the encoded protein can be expressed recombinantly and the activity of the protein can be determined.

As used herein, a “biologically active portion” of a MKP protein includes a fragment of a MKP protein which participates in an interaction between a MKP molecule and a non-MKP molecule. Biologically active portions of a MKP protein include peptides comprising amino acid sequences sufficiently homologous to or derived from the amino acid sequence of the MKP protein, e.g., the amino acid sequence shown in SEQ ID NO:2, which include fewer amino acids than the full length MKP protein, and exhibit at least one activity of a MKP protein. Typically, biologically active portions comprise a domain or motif with at least one activity of the MKP protein, e.g., the ability to modulate (e.g., inactivate, e.g., via dephosphorylation) enzymes involved in signaling pathways, e.g., MAP kinases, and the ability to monitor, treat and/or diagnose disorders in which signaling pathways which MKP molecules of the invention are capable of modulating are involved (e.g., metabolic disorders, in which the insulin signaling pathways, e.g., is involved). A biologically active portion of a MKP protein can be a polypeptide which is, for example, 10, 25, 50, 100, 200 or more amino acids in length. Biologically active portions of a MKP protein can be used as targets for developing agents which modulate a MKP mediated activity.

Calculations of homology or sequence identity (the terms “homology” and “identity” are used interchangeably herein) between sequences are performed as follows:

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90%, 100% of the length of the reference sequence (e.g., when aligning a second sequence to the mouse MKP3 amino acid sequence of SEQ ID NO:2 having 381 amino acid residues, at least 114, preferably at least 152, more preferably at least 190, even more preferably at least 228, and even more preferably at least 266, 304, or 342 amino acid residues are aligned; likewise, e.g., when aligning a second sequence to the mouse MKP4 amino acid sequence of SEQ ID NO:5 having 452 amino acid residues, at least 135, preferably at least 180, more preferably at least 226, even more preferably at least 271, and even more preferably at least 316, 361, or 406 amino acid residues are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (1970) J. Mol. Biol. 48:444-453 algorithm which has been incorporated into the GAP program in the GCG software package (available at http://www.gcg.com), using either a BLOSUM 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the invention) are a BLOSUM 62 scoring matrix with a gap penalty of 12, a gap extend penalty of 4, and a frameshift gap penalty of 5.

The percent identity between two amino acid or nucleotide sequences can be determined using the algorithm of Meyers and Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul et al. (1990) J. Mol. Biol. 215:403-10. BLAST nucleotide searches can be performed with the NBLAST program, score=100, wordlength=12 to obtain nucleotide sequences homologous to MKP nucleic acid molecules of the invention. BLAST protein searches can be performed with the XBLAST program, score=50, wordlength=3 to obtain amino acid sequences homologous to MKP protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. See http:/www.ncbi.nlm.nih.gov.

Particular MKP polypeptides of the invention have an amino acid sequence substantially identical to the amino acid sequence of SEQ ID NO:2 and/or SEQ ID NO:5. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 60%, or 65% identity, likely 75% identity, more likely 85%, 90%. 91%, 92%, 93%, 94%, 95%, 96%, 97%, 98% or 99% identity to SEQ ID NO:2 and/or SEQ ID NO:5 are termed substantially identical.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 60%, or 65% identity, likely 75% identity, more likely 85%, 90%. 91%, 92%, 93%, 94%, 95%, 96%, 97% 98% or 99% identity to SEQ ID NO:1 and/or SEQ ID NO:4 are termed substantially identical.

“Misexpression or aberrant expression”, as used herein, refers to a non-wild type pattern of gene expression, at the RNA or protein level. It includes: expression at non-wild type levels, i.e., over or under expression; a pattern of expression that differs from wild type in terms of the time or stage at which the gene is expressed, e.g., increased or decreased expression (as compared with wild type) at a predetermined developmental period or stage; a pattern of expression that differs from wild type in terms of decreased expression (as compared with wild type) in a predetermined cell type or tissue type; a pattern of expression that differs from wild type in terms of the splicing size, amino acid sequence, post-transitional modification, or biological activity of the expressed polypeptide; a pattern of expression that differs from wild type in terms of the effect of an environmental stimulus or extracellular stimulus on expression of the gene, e.g., a pattern of increased or decreased expression (as compared with wild type) in the presence of an increase or decrease in the strength of the stimulus.

“Subject”, as used herein, can refer to a mammal, e.g., a human, or to an experimental or animal or disease model. The subject can also be a non-human animal, e.g., a horse, cow, goat, or other domestic animal.

A “purified preparation of cells”, as used herein, refers to, in the case of plant or animal cells, an in vitro preparation of cells and not an entire intact plant or animal. In the case of cultured cells or microbial cells, it consists of a preparation of at least 10% and more preferably 50% of the subject cells.

Various aspects of the invention are described in further detail below.

Isolated Nucleic Acid Molecules

In one aspect, the invention provides, an isolated or purified, nucleic acid molecule that encodes a MKP polypeptide described herein, e.g., a full length MKP protein or a fragment thereof, e.g., a biologically active portion of MKP protein. Also included is a nucleic acid fragment suitable for use as a hybridization probe, which can be used, e.g., to identify a nucleic acid molecule encoding a polypeptide of the invention, MKP mRNA, and fragments suitable for use as primers, e.g., PCR primers for the amplification or mutation of nucleic acid molecules.

In one embodiment, an isolated nucleic acid molecule of the invention includes the nucleotide sequence shown in SEQ ID NO:1, or a portion of this nucleotide sequence. In one embodiment, the nucleic acid molecule includes sequences encoding the mouse MKP3 protein (i.e., “the coding region” of SEQ ID NO:1, as shown in SEQ ID NO:3), as well as 5′ untranslated sequences (nucleotides 1 to 279 of SEQ ID NO:1) and 3′ untranslated sequences (nucleotides 1423-2650 of SEQ ID NO:1). Alternatively, the nucleic acid molecule can include only the coding region of SEQ ID NO:1 (e.g., SEQ ID NO:3) and, e.g., no flanking sequences which normally accompany the subject sequence. In another embodiment, the nucleic acid molecule encodes a sequence corresponding to a fragment of the protein from amino acid 206 to about amino acid 346 of SEQ ID NO:2.

In another embodiment, an isolated nucleic acid molecule of the invention includes the nucleotide sequence shown in SEQ ID NO:4, or a portion of this nucleotide sequence. In one embodiment, the nucleic acid molecule includes sequences encoding the mouse MKP4 protein (i.e., “the coding region” of SEQ ID NO:4, as shown in SEQ ID NO:6), as well as 5′ untranslated sequences (nucleotides 1 to 253 of SEQ ID NO:4) and 3′ untranslated sequences (nucleotides 1610-2741 of SEQ ID NO:4). Alternatively, the nucleic acid molecule can include only the coding region of SEQ ID NO:4 (e.g., SEQ ID NO:6) and, e.g., no flanking sequences which normally accompany the subject sequence. In another embodiment, the nucleic acid molecule encodes a sequence corresponding to a fragment of the protein from amino acid 271 to about amino acid 411 of SEQ ID NO:2.

In another embodiment, an isolated nucleic acid molecule of the invention includes a nucleic acid molecule which is a complement of the nucleotide sequence shown in SEQ ID NO:1, SEQ ID NO:3, SEQ ID NO:4, SEQ ID NO:6, or a portion of any of these nucleotide sequences. In other embodiments, the nucleic acid molecule of the invention is sufficiently complementary to the nucleotide sequence shown in SEQ ID NO:1, SEQ ID NO:3, SEQ ID NO:4, SEQ ID NO:6 such that it can hybridize to the nucleotide sequence shown in SEQ ID NO:1, SEQ ID NO:3, SEQ ID NO:4, SEQ ID NO:6, thereby forming a stable duplex.

In one embodiment, an isolated nucleic acid molecule of the invention includes a nucleotide sequence which is at least about: 60%, 65%, 70%, 75%, 80%, 85%, 90%, 91%, 92%, 93%, 94%, 95%, 96%, 97%, 98%, 99%, or more homologous to the entire length of the nucleotide sequence shown in SEQ ID NO:1 or SEQ ID NO:3, the nucleotide sequence shown in SEQ ID NO:4 or SEQ ID NO:6, or a portion, preferably of the same length, of any of these nucleotide sequences.

MKP Family Member Nucleic Acid Fragments

A nucleic acid molecule of the invention can include only a portion of the nucleic acid sequence of SEQ ID NO:1 or SEQ ID NO:4. For example, such a nucleic acid molecule can include a fragment which can be used as a probe or primer or a fragment encoding a portion of a MKP protein, e.g., an immunogenic or biologically active portion of a MKP protein. A fragment can comprise those nucleotides of SEQ ID NO:1 or SEQ ID NO:4 which encode a dual specificity phosphatase, catalytic domain of the MKP family member molecules of the invention. The nucleotide sequence determined from the cloning of the MKP gene allows for the generation of probes and primers designed for use in identifying and/or cloning other MKP family members, or fragments thereof, as well as MKP homologs, or fragments thereof, from other species.

In another embodiment, a nucleic acid includes a nucleotide sequence that includes part, or all, of the coding region and extends into either (or both) the 5′ or 3′ noncoding region. Other embodiments include a fragment which includes a nucleotide sequence encoding an amino acid fragment described herein. Nucleic acid fragments can encode a specific domain or site described herein or fragments thereof, particularly fragments thereof which are at least 100 amino acids in length. Fragments also include nucleic acid sequences corresponding to specific amino acid sequences described above or fragments thereof (e.g., fragments corresponding to the open reading frames of MKP3 and MKP4 (e.g., SEQ ID NO:3 and SEQ ID NO:6), encoding MKP3 and MKP4, respectively (e.g., SEQ ID NO:2 and SEQ ID NO:5)). Nucleic acid fragments should not to be construed as encompassing those fragments that may have been disclosed prior to the invention.

A nucleic acid fragment can include a sequence corresponding to a domain, region, or functional site described herein. A nucleic acid fragment can also include one or more domain, region, or functional site described herein. Thus, for example, a MKP nucleic acid fragment can include a sequence corresponding to a dual specificity phosphatase, catalytic domain, as described herein.

MKP probes and primers are provided. Typically a probe/primer is an isolated or purified oligonucleotide. The oligonucleotide typically includes a region of nucleotide sequence that hybridizes under stringent conditions to at least about 7, 12 or 15, preferably about 20 or 25, more preferably about 30, 35, 40, 45, 50, 55, 60, 65, or 75 consecutive nucleotides of a sense or antisense sequence of SEQ ID NO:1 or SEQ ID NO:4, or of a naturally occurring allelic variant or mutant of SEQ ID NO:1 or SEQ ID NO:4.

In a preferred embodiment the nucleic acid is a probe which is at least 5 or 10, and less than 200, more preferably less than 100, or less than 50, base pairs in length. It should be identical, or differ by 1, or less than 5 or 10 bases, from a sequence disclosed herein. If alignment is needed for this comparison the sequences should be aligned for maximum homology. “Looped” out sequences from deletions or insertions, or mismatches, are considered differences.

A probe or primer can be derived from the sense or anti-sense strand of a nucleic acid which encodes a dual specificity phosphatase, catalytic domain, or a rhodanese-like domain.

In another embodiment a set of primers is provided, e.g., primers suitable for use in PCR, which can be used to amplify a selected region of a MKP sequence, e.g., a domain, region, site or other sequence described herein. The primers should be at least 5, 10, or 50 base pairs in length and less than 100, or less than 200, base pairs in length. The primers should be identical, or differ by one base from a sequence disclosed herein or from a naturally occurring variant. For example, primers suitable for amplifying all or a portion of any of the following regions are provided: a dual specificity phosphatase, catalytic domain, from about amino acid 206 to about amino acid 346 of SEQ ID NO:2 (and from about amino acid 271 to about amino acid 411 of SEQ ID NO:5) or a rhodanese-like domain from about amino acid 19 to about amino acid 142 of SEQ ID NO:2 (and from about amino acid 7 to about amino acid 201 of SEQ ID NO:5).

A nucleic acid fragment can encode an epitope bearing region of a polypeptide described herein.

A nucleic acid fragment encoding a “biologically active portion of a MKP polypeptide” can be prepared by isolating a portion of the nucleotide sequence of SEQ ID NO:1 or SEQ ID NO:4, which encodes a polypeptide having a MKP biological activity (e.g., the biological activities of the MKP proteins are described herein), expressing the encoded portion of the MKP protein (e.g., by recombinant expression in vitro) and assessing the activity of the encoded portion of the MKP protein. For example, a nucleic acid fragment encoding a biologically active portion of MKP includes a dual specificity phosphatase, catalytic domain, e.g., amino acid residues about 206 to about amino acid 346 of SEQ ID NO:2 (amino acid residues from about 271 to about 411 of SEQ ID NO:5). A nucleic acid fragment encoding a biologically active portion of a MKP polypeptide, can comprise a nucleotide sequence which is greater than 300 or more nucleotides in length.

In preferred embodiments, a nucleic acid includes a nucleotide sequence which is about 300, 400, 500, 600, 700, 800, 900, 1000, 1100, 1200, 1300, 1400, 1500, 1600, 1700, 1800, 1900, 2000, 2100, 2200, 2300, 2400, 2500, 2600, or more nucleotides in length and hybridizes under stringent hybridization conditions to a nucleic acid molecule of SEQ ID NO:1 or SEQ ID NO:4.

MKP Nucleic Acid Variants

The invention further encompasses nucleic acid molecules that differ from the nucleotide sequence shown in SEQ ID NO:1 or SEQ ID NO:4. Such differences can be due to degeneracy of the genetic code (and result in a nucleic acid which encodes the same MKP proteins as those encoded by the nucleotide sequence disclosed herein. In another embodiment, an isolated nucleic acid molecule of the invention has a nucleotide sequence encoding a protein having an amino acid sequence which differs, by at least 1, but less than 5, 10, 20, 50, or 100 amino acid residues that shown in SEQ ID NO:2 or SEQ ID NO:5. If alignment is needed for this comparison the sequences should be aligned for maximum homology. “Looped” out sequences from deletions or insertions, or mismatches, are considered differences.

Nucleic acids of the invention can be chosen for having codons, which are preferred, or non-preferred, for a particular expression system. E.g., the nucleic acid can be one in which at least one codon, at preferably at least 10%, or 20% of the codons has been altered such that the sequence is optimized for expression in E. coli, yeast, human, insect, or CHO cells.

Nucleic acid variants can be naturally occurring, such as allelic variants (same locus), homologs (different locus), and orthologs (different organism) or can be non-naturally occurring. Non-naturally occurring variants can be made by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared in the encoded product).

In a preferred embodiment, the nucleic acid differs from that of SEQ ID NO:1, 3, 4, or 6, e.g., as follows: by at least one but less than 10, 20, 30, or 40 nucleotides; at least one but less than 1%, 5%, 10% or 20% of the nucleotides in the subject nucleic acid. If necessary for this analysis the sequences should be aligned for maximum homology. “Looped” out sequences from deletions or insertions, or mismatches, are considered differences.

Orthologs, homologs, and allelic variants can be identified using methods known in the art. These variants comprise a nucleotide sequence encoding a polypeptide that is 50%, at least about 55%, typically at least about 70-75%, more typically at least about 80-85%, and most typically at least about 90-95% or more identical to the nucleotide sequence shown in SEQ ID NO:2 or a fragment of this sequence. Such nucleic acid molecules can readily be identified as being able to hybridize under stringent conditions, to the nucleotide sequence shown in SEQ ID NO 2 or a fragment of the sequence. Nucleic acid molecules corresponding to orthologs, homologs, and allelic variants of the MKP cDNAs of the invention can further be isolated by mapping to the same chromosome or locus as the MKP gene.

Preferred variants include those that are correlated with the ability to modulate (e.g., inactivate, e.g., via dephosphorylation) enzymes involved in signaling pathways, e.g., MAP kinases, and the ability to monitor, treat and/or diagnose disorders in which signaling pathways which MKP molecules of the invention are capable of modulating are involved (e.g., metabolic disorders, in which the insulin signaling pathways, e.g., is involved)

Allelic variants of MKP, e.g., human MKP, include both functional and non-functional proteins. Functional allelic variants are naturally occurring amino acid sequence variants of the MKP protein within a population that maintain the ability to, for instance, modulate (e.g., inactivate, e.g., via dephosphorylation) enzymes involved in signaling pathways, e.g., MAP kinases, and the ability to monitor, treat and/or diagnose disorders in which signaling pathways which MKP molecules of the invention are capable of modulating are involved (e.g., metabolic disorders, in which the insulin signaling pathways, e.g., is involved)

Functional allelic variants will typically contain only conservative substitution of one or more amino acids of SEQ ID NO:2, or substitution, deletion or insertion of non-critical residues in non-critical regions of the protein. Non-functional allelic variants are naturally-occurring amino acid sequence variants of the MKP, e.g., human MKP, protein within a population that do not have the ability to, for instance, modulate (e.g., inactivate, e.g., via dephosphorylation) enzymes involved in signaling pathways, e.g., MAP kinases, and the ability to monitor, treat and/or diagnose disorders in which signaling pathways which MKP molecules of the invention are capable of modulating are involved (e.g., metabolic disorders, in which the insulin signaling pathways, e.g., is involved)

Non-functional allelic variants will typically contain a non-conservative substitution, a deletion, or insertion, or premature truncation of the amino acid sequence of SEQ ID NO:2, or a substitution, insertion, or deletion in critical residues or critical regions of the protein.

Moreover, nucleic acid molecules encoding other MKP family members and, thus, which have a nucleotide sequence which differs from the MKP sequences of SEQ ID NO:1 or SEQ ID NO:4 are intended to be within the scope of the invention.

Antesense Nucleic Acid Molecules, Ribozymes and Modified MKP Nucleic Acid Molecules

In another aspect, the invention features, an isolated nucleic acid molecule which is antisense to MKP. An “antisense” nucleic acid can include a nucleotide sequence which is complementary to a “sense” nucleic acid encoding a protein, e.g., complementary to the coding strand of a double-stranded cDNA molecule or complementary to an mRNA sequence. The antisense nucleic acid can be complementary to an entire MKP coding strand, or to only a portion thereof (e.g., the coding region of human MKP corresponding to SEQ ID NO:3). In another embodiment, the antisense nucleic acid molecule is antisense to a “noncoding region” of the coding strand of a nucleotide sequence encoding MKP (e.g., the 5′ and 3′ untranslated regions).

An antisense nucleic acid can be designed such that it is complementary to the entire coding region of MKP mRNA, but more preferably is an oligonucleotide which is antisense to only a portion of the coding or noncoding region of MKP mRNA. For example, the antisense oligonucleotide can be complementary to the region surrounding the translation start site of MKP mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length.

An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. The antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject (e.g., by direct injection at a tissue site), or generated in situ such that they hybridize with or bind to cellular mRNA and/or genomic DNA encoding a MKP protein to thereby inhibit expression of the protein, e.g., by inhibiting transcription and/or translation. Alternatively, antisense nucleic acid molecules can be modified to target selected cells and then administered systemically. For systemic administration, antisense molecules can be modified such that they specifically or selectively bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987)FEBS Lett. 215:327-330).

In still another embodiment, an antisense nucleic acid of the invention is a ribozyme. A ribozyme having specificity for a MKP-encoding nucleic acid can include one or more sequences complementary to the nucleotide sequence of a MKP cDNA disclosed herein (i.e., SEQ ID NO:1 or SEQ ID NO:4), and a sequence having known catalytic sequence responsible for mRNA cleavage (see U.S. Pat. No. 5,093,246 or Haselhoff and Gerlach (1988) Nature 334:585-591). For example, a derivative of a Tetrahymena L-19 IVS RNA can be constructed in which the nucleotide sequence of the active site is complementary to the nucleotide sequence to be cleaved in a MKP-encoding mRNA. See, e.g., Cech et al. U.S. Pat. No. 4,987,071; and Cech et al. U.S. Pat. No. 5,116,742. Alternatively, MKP mRNA can be used to select a catalytic RNA having a specific ribonuclease activity from a pool of RNA molecules. See, e.g., Bartel and Szostak (1993) Science 261:1411-1418.

MKP gene expression can be inhibited by targeting nucleotide sequences complementary to the regulatory region of the MKP (e.g., the MKP promoter and/or enhancers) to form triple helical structures that prevent transcription of the MKP gene in target cells. See generally, Helene (1991) Anticancer Drug Des. 6:569-84; Helene (1992) Ann. N.Y. Acad. Sci. 660:27-36; and Maher (1992) Bioassays 14:807-15. The potential sequences that can be targeted for triple helix formation can be increased by creating a so-called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

The invention also provides detectably labeled oligonucleotide primer and probe molecules. Typically, such labels are chemiluminescent, fluorescent, radioactive, or colorimetric.

A MKP nucleic acid molecule can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup et al. (1996) Bioorganic & Medicinal Chemistry 4: 5-23). As used herein, the terms “peptide nucleic acid” or “PNA” refers to a nucleic acid mimic, e.g., a DNA mimic, in which the deoxyribose phosphate backbone is replaced by a pseudopeptide backbone and only the four natural nucleobases are retained. The neutral backbone of a PNA can allow for specific hybridization to DNA and RNA under conditions of low ionic strength. The synthesis of PNA oligomers can be performed using standard solid phase peptide synthesis protocols as described in Hyrup et al. (1996) supra; Perry-O'Keefe et al. (1996) Proc. Natl. Acad. Sci. 93: 14670-675.

PNAs of MKP nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of MKP nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup et al. (1996) supra)); or as probes or primers for DNA sequencing or hybridization (Hyrup et al. (1996) supra; Perry-O'Keefe supra).

In other embodiments, the oligonucleotide can include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. W088/09810) or the blood-brain barrier (see, e.g., PCT Publication No. W089/10134). In addition, oligonucleotides can be modified with hybridization-triggered cleavage agents (see, e.g., Krol et al. (1988) Bio-Techniques 6:958-976) or intercalating agents. (see, e.g., Zon (1988) Pharm. Res. 5:539-549). To this end, the oligonucleotide can be conjugated to another molecule, (e.g., a peptide, hybridization triggered cross-linking agent, transport agent, or hybridization-triggered cleavage agent).

The invention also includes molecular beacon oligonucleotide primer and probe molecules having at least one region which is complementary to a MKP nucleic acid of the invention, two complementary regions one having a fluorophore and one a quencher such that the molecular beacon is useful for quantitating the presence of the MKP nucleic acid of the invention in a sample. Molecular beacon nucleic acids are described, for example, in Lizardi et al., U.S. Pat. No. 5,854,033; Nazarenko et al., U.S. Pat. No. 5,866,336, and Livak et al., U.S. Pat. 5,876,930.

Isolated MKP Polypeptides

In another aspect, the invention features, an isolated MKP protein, or fragment, e.g., a biologically active portion, for use as immunogens or antigens to raise or test (or more generally to bind) anti-MKP antibodies. MKP protein can be isolated from cells or tissue sources using standard protein purification techniques. MKP protein or fragments thereof can be produced by recombinant DNA techniques or synthesized chemically.

Polypeptides of the invention include those which arise as a result of the existence of multiple genes, alternative transcription events, alternative RNA splicing events, and alternative translational and post-translational events. The polypeptide can be expressed in systems, e.g., cultured cells, which result in substantially the same post-translational modifications present when the polypeptide is expressed in a native cell, or in systems which result in the alteration or omission of post-translational modifications, e.g., glycosylation or cleavage, present in a native cell.

In a preferred embodiment, a MKP polypeptide has one or more of the following characteristics: a) it has the ability to the ability (1) modulate (e.g., inactivate, e.g., via dephosphorylation) enzymes involved in signaling pathways, e.g., MAP kinases; (2) reverse the effects of the activities of enzymes (e.g., MEKs) involved in signaling pathways; (3) modulate (e.g., inhibit) pathways via the modulation (e.g., inhibition) of components of said pathways (e.g., signal transduction pathways (e.g., those involved in cellular growth, motigenesis, and differentiation)); (4) monitor, treat and/or diagnose disorders in which signaling pathways which MKP molecules of the invention are capable of modulating are involved (e.g., metabolic disorders, in which the insulin signaling pathways, e.g., is involved); (5) modulate (e.g., prevent) cellular differentiation (e.g., the differentiation of preadipocytes into adipocytes); and (6) modulate glycogen synthesis, e.g., the differentiation or stimulation thereof.

b) it has a molecular weight of 42.4 kDa or 48.7 kDa (mouse MKP3 and mouse MKP4, respectively), e.g., a deduced molecular weight, preferably ignoring any contribution of post translational modifications, amino acid composition or other physical characteristic of a MKP polypeptide, e.g., a polypeptide of SEQ ID NO:2;

c) it has an overall sequence similarity of at least 60%, preferably at least 70%, more preferably at least 80, 90, or 95%, with a polypeptide of SEQ ID NO:2 or SEQ ID NO:5;

d) it is expressed in at least insulin-related (e.g., insulin-responsive) tissues in mice models of obesity (i.e., compared to in wild type mice), for example, in adipose tissue and liver;

e) it has a dual specificity phosphatase, catalytic domain which is preferably about 70%, 80%, 90% or 95% identical to amino acid residues about 206 to about amino acid 346 of SEQ ID NO:2 (about amino acid residues 271-411 of SEQ ID NO:5); and

f) it has a rhodanese-like domain which is preferably about 70%, 80%, 90% or 95% identical to amino acid residues about 19-142 of SEQ ID NO:2 (about amino acid residues 7-201 of SEQ ID NO:5).

In a preferred embodiment the MKP protein, or fragment thereof, differs from the corresponding sequence in SEQ ID NO:2. In one embodiment it differs by at least one but by less than 15, 10 or 5 amino acid residues. In another it differs from the corresponding sequence in SEQ ID NO:2 by at least one residue but less than 20%, 15%, 10% or 5% of the residues in it differ from the corresponding sequence in SEQ ID NO:2. (If this comparison requires alignment the sequences should be aligned for maximum homology. “Looped” out sequences from deletions or insertions, or mismatches, are considered differences.) The differences are, preferably, differences or changes at a non-essential residue or a conservative substitution. In a preferred embodiment the differences are not in the dual specificity phosphatase, catalytic domain at about residue 206 to about amino acid 346 of SEQ ID NO:2 (about residues 271-411 of SEQ ID NO:5).

Other embodiments include a protein that contains one or more changes in amino acid sequence, e.g., a change in an amino acid residue which is not essential for activity. Such MKP proteins differ in amino acid sequence from SEQ ID NO:2 or SEQ ID NO:5, yet retain biological activity.

In one embodiment, the protein includes an amino acid sequence at least about 60%, 65%, 70%, 75%, 80%, 85%, 90%, 95%, 98% or more homologous to SEQ ID NO:2 or SEQ ID NO:5.

In one embodiment, a biologically active portion of a MKP protein includes a dual specificity phosphatase, catalytic domain. Moreover, other biologically active portions, in which other regions of the protein are deleted, can be prepared by recombinant techniques and evaluated for one or more of the functional activities of a native MKP protein.

In a preferred embodiment, the MKP protein has an amino acid sequence shown in SEQ ID NO:2 or SEQ ID NO:5. In other embodiments, the MKP protein is sufficiently or substantially identical to SEQ ID NO:2 or SEQ ID NO:5. In yet another embodiment, the MKP protein is sufficiently or substantially identical to SEQ ID NO:2 or SEQ ID NO:5 and retains the functional activity of the protein of SEQ ID NO:2 or SEQ ID NO:5, as described in detail in the subsections above.

MKP Chimeric or Fusion Proteins

In another aspect, the invention provides MKP chimeric or fusion proteins. As used herein, a MKP “chimeric protein” or “fusion protein” includes a MKP polypeptide linked to a non-MKP polypeptide. A “non-MKP polypeptide” refers to a polypeptide having an amino acid sequence corresponding to a protein which is not substantially homologous to the MKP protein, e.g., a protein which is different from the MKP protein and which is derived from the same or a different organism. The MKP polypeptide of the fusion protein can correspond to all or a portion e.g., a fragment described herein of a MKP amino acid sequence. In a preferred embodiment, a MKP fusion protein includes at least one (or two) biologically active portion of a MKP protein. The non-MKP polypeptide can be fused to the N-terminus or C-terminus of the MKP polypeptide.

The fusion protein can include a moiety which has a high affinity for a ligand. For example, the fusion protein can be a GST-MKP fusion protein in which the MKP sequences are fused to the C-terminus of the GST sequences. Such fusion proteins can facilitate the purification of recombinant MKP. Alternatively, the fusion protein can be a MKP protein containing a heterologous signal sequence at its N-terminus. In certain host cells (e.g., mammalian host cells), expression and/or secretion of MKP can be increased through use of a heterologous signal sequence.

Fusion proteins can include all or a part of a serum protein, e.g., a portion of an immunoglobulin (e.g., IgG, IgA, or IgE), e.g., an Fc region and/or the hinge C1 and C2 sequences of an immunoglobulin or human serum albumin.

The MKP fusion proteins of the invention can be incorporated into pharmaceutical compositions and administered to a subject in vivo. The MKP fusion proteins can be used to affect the bioavailability of a MKP substrate. MKP fusion proteins can be useful therapeutically for the treatment of disorders caused by, for example, (i) aberrant modification or mutation of a gene encoding a MKP protein; (ii) mis-regulation of the MKP gene; and (iii) aberrant post-translational modification of a MKP protein.

Moreover, the MKP-fusion proteins of the invention can be used as immunogens to produce anti-MKP antibodies in a subject, to purify MKP ligands and in screening assays to identify molecules which inhibit the interaction of MKP with a MKP substrate.

Expression vectors are commercially available that already encode a fusion moiety (e.g., a GST polypeptide). A MKP-encoding nucleic acid can be cloned into such an expression vector such that the fusion moiety is linked in-frame to the MKP protein.

Variants of MKP Proteins

In another aspect, the invention also features a variant of a MKP polypeptide, e.g., which functions as an agonist (mimetics) or as an antagonist. Variants of the MKP proteins can be generated by mutagenesis, e.g., discrete point mutation, the insertion or deletion of sequences or the truncation of a MKP protein. An agonist of the MKP proteins can retain substantially the same, or a subset, of the biological activities of the naturally occurring form of a MKP protein. An antagonist of a MKP protein can inhibit one or more of the activities of the naturally occurring form of the MKP protein by, for example, competitively modulating a MKP-mediated activity of a MKP protein. Thus, specific biological effects can be elicited by treatment with a variant of limited function. Preferably, treatment of a subject with a variant having a subset of the biological activities of the naturally occurring form of the protein has fewer side effects in a subject relative to treatment with the naturally occurring form of the MKP protein.

Variants of a MKP protein can be identified by screening combinatorial libraries of mutants, e.g., truncation mutants, of a MKP protein for agonist or antagonist activity.

Libraries of fragments e.g., N terminal, C terminal, or internal fragments, of a MKP protein coding sequence can be used to generate a variegated population of fragments for screening and subsequent selection of variants of a MKP protein.

Variants in which a cysteine residues is added or deleted or in which a residue which is glycosylated is added or deleted are particularly preferred.

Methods for screening gene products of combinatorial libraries made by point mutations or truncation, and for screening cDNA libraries for gene products having a selected property are known in the art. Recursive ensemble mutagenesis (REM), a new technique which enhances the frequency of functional mutants in the libraries, can be used in combination with the screening assays to identify MKP variants (Arkin and Yourvan (1992) Proc. Natl. Acad. Sci. USA 89:7811-7815; Delgrave et al. (1993) Protein Engineering 6:327-331).

Cell based assays can be exploited to analyze a variegated MKP library. For example, a library of expression vectors can be transfected into a cell line, e.g., a cell line, which ordinarily responds to MKP in a substrate-dependent manner. The transfected cells are then contacted with MKP and the effect of the expression of the mutant on signaling by the MKP substrate can be detected, e.g., by measuring phosphatase activity. Plasmid DNA can then be recovered from the cells which score for inhibition, or alternatively, potentiation of signaling by the MKP substrate, and the individual clones further characterized.

In another aspect, the invention features a method of making a MKP polypeptide, e.g., a peptide having a non-wild type activity, e.g., an antagonist, agonist, or super agonist of a naturally occurring MKP polypeptide, e.g., a naturally occurring MKP polypeptide. The method includes altering the sequence of a MKP polypeptide, e.g., altering the sequence, e.g., by substitution or deletion of one or more residues of a non-conserved region, a domain or residue disclosed herein, and testing the altered polypeptide for the desired activity.

In another aspect, the invention features a method of making a fragment or analog of a MKP polypeptide a biological activity of a naturally occurring MKP polypeptide. The method includes altering the sequence, e.g., by substitution or deletion of one or more residues, of a MKP polypeptide, e.g., altering the sequence of a non-conserved region, or a domain or residue described herein, and testing the altered polypeptide for the desired activity.

Anti-MKP Antibodies

In another aspect, the invention provides an anti-MKP antibody. The term “antibody” as used herein refers to an immunoglobulin molecule or immunologically active portion thereof, i.e., an antigen-binding portion. Examples of immunologically active portions of immunoglobulin molecules include scFV and dcFV fragments, Fab and F(ab')₂ fragments which can be generated by treating the antibody with an enzyme such as papain or pepsin, respectively.

The antibody can be a polyclonal, monoclonal, recombinant, e.g., a chimeric or humanized, fully human, non-human, e.g., murine, or single chain antibody. In a preferred embodiment it has effector function and can fix complement. The antibody can be coupled to a toxin or imaging agent.

A full-length MKP protein or, antigenic peptide fragment of MKP can be used as an immunogen or can be used to identify anti-MKP antibodies made with other immunogens, e.g., cells, membrane preparations, and the like. The antigenic peptide of MKP should include at least 8 amino acid residues of the amino acid sequence shown in SEQ ID NO:2 and encompasses an epitope of MKP. Preferably, the antigenic peptide includes at least 10 amino acid residues, more preferably at least 15 amino acid residues, even more preferably at least 20 amino acid residues, and most preferably at least 30 amino acid residues.

Antibodies reactive with, or specific or selective for, any of these regions, or other regions or domains described herein are provided.

Preferred epitopes encompassed by the antigenic peptide are regions of MKP located on the surface of the protein, e.g., hydrophilic regions, as well as regions with high antigenicity. For example, an Emini surface probability analysis of the human MKP protein sequence can be used to indicate the regions that have a particularly high probability of being localized to the surface of the MKP protein and are thus likely to constitute surface residues useful for targeting antibody production.

In a preferred embodiment the antibody can bind to the extracellular portion of the MKP protein, e.g., it can bind to a whole cell which expresses the MKP protein. In another embodiment, the antibody binds an intracellular portion of the MKP protein.

In a preferred embodiment the antibody binds an epitope on any domain or region on MKP proteins described herein.

Additionally, chimeric, humanized, and completely human antibodies are also within the scope of the invention. Chimeric, humanized, but most preferably, completely human antibodies are desirable for applications which include repeated administration, e.g., therapeutic treatment of human patients, and some diagnostic applications.

Chimeric and humanized monoclonal antibodies, comprising both human and non-human portions, can be made using standard recombinant DNA techniques. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

Completely human antibodies are particularly desirable for therapeutic treatment of human patients. Such antibodies can be produced using transgenic mice that are incapable of expressing endogenous immunoglobulin heavy and light chains genes, but which can express human heavy and light chain genes. See, for example, Lonberg and Huszar (1995) Int. Rev. Immunol. 13:65-93); and U.S. Pat. Nos. 5,625,126; 5,633,425; 5,569,825; 5,661,016; and 5,545,806. In addition, companies such as Abgenix, Inc. (Fremont, Calif. and Medarex, Inc. (Princeton, N.J.), can be engaged to provide human antibodies directed against a selected antigen using technology similar to that described above.

Completely human antibodies that recognize a selected epitope can be generated using a technique referred to as “guided selection.” In this approach a selected non-human monoclonal antibody, e.g., a murine antibody, is used to guide the selection of a completely human antibody recognizing the same epitope. This technology is described by Jespers et al. (1994) Bio/Technology 12:899-903).

The anti-MKP antibody can be a single chain antibody. A single-chain antibody (scFV) can be engineered as described in, for example, Colcher et al. (1999) Ann. N Y Acad. Sci. 880:263-80; and Reiter (1996) Clin. Cancer Res. 2:245-52. The single chain antibody can be dimerized or multimerized to generate multivalent antibodies having specificities for different epitopes of the same target MKP protein.

In a preferred embodiment, the antibody has reduced or no ability to bind an Fc receptor. For example, it is an isotype or subtype, fragment or other mutant, which does not support binding to an Fc receptor, e.g., it has a mutagenized or deleted Fc receptor binding region.

An antibody (or fragment thereof) may be conjugated to a therapeutic moiety such as a cytotoxin, a therapeutic agent or a radioactive ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, maytansinoids, e.g., maytansinol (see U.S. Pat. No. 5,208,020), CC-1065 (see U.S. Pat. Nos. 5,475,092, 5,585,499, 5,846,545) and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, CC-1065, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine, vinblastine, taxol and maytansinoids). Radioactive ions include, but are not limited to iodine, yttrium and praseodymium.

The conjugates of the invention can be used for modifying a given biological response, the therapeutic moiety is not to be construed as limited to classical chemical therapeutic agents. For example, the therapeutic moiety may be a protein or polypeptide possessing a desired biological activity. Such proteins may include, for example, a toxin such as abrin, ricin A, pseudomonas exotoxin, or diphtheria toxin; a protein such as tumor necrosis factor, α-interferon, β-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Alternatively, an antibody can be conjugated to a second antibody to form an antibody heteroconjugate as described by Segal in U.S. Pat. No. 4,676,980.

An anti-MKP antibody (e.g., monoclonal antibody) can be used to isolate MKP by standard techniques, such as affinity chromatography or immunoprecipitation. Moreover, an anti-MKP antibody can be used to detect MKP protein (e.g., in a cellular lysate or cell supernatant) in order to evaluate the abundance and pattern of expression of the protein. Anti-MKP antibodies can be used diagnostically to monitor protein levels in tissue as part of a clinical testing procedure, e.g., to determine the efficacy of a given treatment regimen. Detection can be facilitated by coupling (i.e., physically linking) the antibody to a detectable substance (i.e., antibody labelling). Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or³H.

In preferred embodiments, an antibody can be made by immunizing with a purified MKP antigen, or a fragment thereof, e.g., a fragment described herein, a membrane associated antigen, tissues, e.g., crude tissue preparations, whole cells, preferably living cells, lysed cells, or cell fractions, e.g., membrane fractions.

Antibodies which bind only a native MKP protein, only denatured or otherwise non-native MKP protein, or which bind both, are within the invention. Antibodies with linear or conformational epitopes are within the invention. Conformational epitopes sometimes can be identified by identifying antibodies which bind to native but not denatured MKP protein.

Recombinant Expression Vectors, Host Cells and Genetically Engineered Cells

In another aspect, the invention includes, vectors, preferably expression vectors, containing a nucleic acid encoding a polypeptide described herein. As used herein, the term “vector” refers to a nucleic acid molecule capable of transporting another nucleic acid to which it has been linked and can include a plasmid, cosmid or viral vector. The vector can be capable of autonomous replication or it can integrate into a host DNA. Viral vectors include, e.g., replication defective retroviruses, adenoviruses and adeno-associated viruses.

A vector can include a MKP nucleic acid in a form suitable for expression of the nucleic acid in a host cell. Preferably the recombinant expression vector includes one or more regulatory sequences operatively linked to the nucleic acid sequence to be expressed. The term “regulatory sequence” includes promoters, enhancers and other expression control elements (e.g., polyadenylation signals). Regulatory sequences include those which direct constitutive expression of a nucleotide sequence, as well as tissue-specific regulatory and/or inducible sequences. The design of the expression vector can depend on such factors as the choice of the host cell to be transformed, the level of expression of protein desired, and the like. The expression vectors of the invention can be introduced into host cells to thereby produce proteins or polypeptides, including fusion proteins or polypeptides, encoded by nucleic acids as described herein (e.g., MKP proteins, mutant forms of MKP proteins, fusion proteins, and the like).

The recombinant expression vectors of the invention can be designed for expression of MKP proteins in prokaryotic or eukaryotic cells. For example, polypeptides of the invention can be expressed in E. coli, insect cells (e.g., using baculovirus expression vectors), yeast cells or mammalian cells. Suitable host cells are discussed further in Goeddel, (1990) Gene Expression Technology: Methods in Enzymology 185, Academic Press, San Diego, Calif. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be used in MKP activity assays, (e.g., direct assays or competitive assays described in detail below), or to generate antibodies specific or selective for MKP proteins. In a preferred embodiment, a fusion protein expressed in a retroviral expression vector of the invention can be used to infect bone marrow cells which are subsequently transplanted into irradiated recipients. The pathology of the subject recipient is then examined after sufficient time has passed (e.g., six weeks).

To maximize recombinant protein expression in E. coli is to express the protein in a host bacteria with an impaired capacity to proteolytically cleave the recombinant protein (Gottesman (1990) Gene Expression Technology: Methods in Enzymology 185, Academic Press, San Diego, Calif. 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

The MKP expression vector can be a yeast expression vector, a vector for expression in insect cells, e.g., a baculovirus expression vector or a vector suitable for expression in mammalian cells.

When used in mammalian cells, the expression vector's control functions are often provided by viral regulatory elements. For example, commonly used promoters are derived from polyoma, Adenovirus 2, cytomegalovirus and Simian Virus 40.

In another embodiment, the recombinant mammalian expression vector is capable of directing expression of the nucleic acid preferentially in a particular cell type (e.g., tissue-specific regulatory elements are used to express the nucleic acid). Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example, the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. Regulatory sequences (e.g., viral promoters and/or enhancers) operatively linked to a nucleic acid cloned in the antisense orientation can be chosen which direct the constitutive, tissue specific or cell type specific expression of antisense RNA in a variety of cell types. The antisense expression vector can be in the form of a recombinant plasmid, phagemid or attenuated virus. For a discussion of the regulation of gene expression using antisense genes see Weintraub et al., (1986) Reviews—Trends in Genetics 1:1.

Another aspect the invention provides a host cell which includes a nucleic acid molecule described herein, e.g., a MKP nucleic acid molecule within a recombinant expression vector or a MKP nucleic acid molecule containing sequences which allow it to homologously recombine into a specific site of the host cell's genome. The terms “host cell” and “recombinant host cell” are used interchangeably herein. Such terms refer not only to the particular subject cell but to the progeny or potential progeny of such a cell. Because certain modifications can occur in succeeding generations due to either mutation or environmental influences, such progeny may not, in fact, be identical to the parent cell, but are still included within the scope of the term as used herein.

A host cell can be any prokaryotic or eukaryotic cell. For example, a MKP protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into host cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation.

A host cell of the invention can be used to produce (i.e., express) a MKP protein. Accordingly, the invention further provides methods for producing a MKP protein using the host cells of the invention. In one embodiment, the method includes culturing the host cell of the invention (into which a recombinant expression vector encoding a MKP protein has been introduced) in a suitable medium such that a MKP protein is produced. In another embodiment, the method further includes isolating a MKP protein from the medium or the host cell.

In another aspect, the invention features, a cell or purified preparation of cells which include a MKP transgene, or which otherwise misexpress MKP. The cell preparation can consist of human or non-human cells, e.g., rodent cells, e.g., mouse or rat cells, rabbit cells, or pig cells. In preferred embodiments, the cell or cells include a MKP transgene, e.g., a heterologous form of a MKP, e.g., a gene derived from humans (in the case of a non-human cell). The MKP transgene can be misexpressed, e.g., overexpressed or underexpressed. In other preferred embodiments, the cell or cells include a gene which misexpresses an endogenous MKP, e.g., a gene the expression of which is disrupted, e.g., a knockout. Such cells can serve as a model for studying disorders which are related to mutated or misexpressed MKP alleles or for use in drug screening.

In another aspect, the invention features, a human cell, e.g., a hematopoietic stem cell, transformed with nucleic acid which encodes a subject MKP polypeptide.

Also provided are cells, preferably human cells, e.g., human hematopoietic or fibroblast cells, in which an endogenous MKP is under the control of a regulatory sequence that does not normally control the expression of the endogenous MKP gene. The expression characteristics of an endogenous gene within a cell, e.g., a cell line or microorganism, can be modified by inserting a heterologous DNA regulatory element into the genome of the cell such that the inserted regulatory element is operably linked to the endogenous MKP gene. For example, an endogenous MKP gene which is “transcriptionally silent,” e.g., not normally expressed, or expressed only at very low levels, can be activated by inserting a regulatory element which is capable of promoting the expression of a normally expressed gene product in that cell. Techniques such as targeted homologous recombinations, can be used to insert the heterologous DNA as described in, e.g., Chappel, U.S. Pat. No. 5,272,071; WO 91/06667, published in May 16, 1991.

Transgenic Animals

The invention provides non-human transgenic animals. Such animals are useful for studying the function and/or activity of a MKP protein and for identifying and/or evaluating modulators of MKP activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA or a rearrangement, e.g., a deletion of endogenous chromosomal DNA, which preferably is integrated into or occurs in the genome of the cells of a transgenic animal. A transgene can direct the expression of an encoded gene product in one or more cell types or tissues of the transgenic animal, other transgenes, e.g., a knockout, reduce expression. Thus, a transgenic animal can be one in which an endogenous MKP gene has been altered by, e.g., by homologous recombination between the endogenous gene and an exogenous DNA molecule introduced into a cell of the animal, e.g., an embryonic cell of the animal, prior to development of the animal.

Intronic sequences and polyadenylation signals can also be included in the transgene to increase the efficiency of expression of the transgene. A tissue-specific regulatory sequence(s) can be operably linked to a transgene of the invention to direct expression of a MKP protein to particular cells. A transgenic founder animal can be identified based upon the presence of a MKP transgene in its genome and/or expression of MKP mRNA in tissues or cells of the animals. A transgenic founder animal can then be used to breed additional animals carrying the transgene. Moreover, transgenic animals carrying a transgene encoding a MKP protein can further be bred to other transgenic animals carrying other transgenes.

MKP proteins or polypeptides can be expressed in transgenic animals or plants, e.g., a nucleic acid encoding the protein or polypeptide can be introduced into the genome of an animal. In preferred embodiments the nucleic acid is placed under the control of a tissue specific promoter, e.g., a milk or egg specific promoter, and recovered from the milk or eggs produced by the animal. Suitable animals are mice, pigs, cows, goats, and sheep.

The invention also includes a population of cells from a transgenic animal, as discussed, e.g., below.

Uses

The nucleic acid molecules, proteins, protein homologs, and antibodies described herein can be used in one or more of the following methods: a) screening assays; b) predictive medicine (e.g., diagnostic assays, prognostic assays, monitoring clinical trials, and pharmacogenetics); and c) methods of treatment (e.g., therapeutic and prophylactic). The isolated nucleic acid molecules of the invention can be used, for example, to express a MKP protein (e.g., via a recombinant expression vector in a host cell in gene therapy applications), to detect a MKP mRNA (e.g., in a biological sample) or a genetic alteration in a MKP gene, and to modulate MKP activity, as described further below. The MKP proteins can be used to treat disorders characterized by insufficient or excessive production of a MKP substrate or production of MKP inhibitors. In addition, the MKP proteins can be used to screen for naturally occurring MKP substrates, to screen for drugs or compounds which modulate MKP activity, as well as to treat disorders characterized by insufficient or excessive production of MKP protein or production of MKP protein forms which have decreased, aberrant or unwanted activity compared to MKP wild type protein (e.g., aberrant or deficient metabolic function or expression). Moreover, the anti-MKP antibodies of the invention can be used to detect and isolate MKP proteins, regulate the bioavailability of MKP proteins, and modulate MKP activity.

A method of evaluating a compound for the ability to interact with, e.g., bind, a subject MKP polypeptide is provided. The method includes: contacting the compound with the subject MKP polypeptide; and evaluating ability of the compound to interact with, e.g., to bind or form a complex with the subject MKP polypeptide. This method can be performed in vitro, e.g., in a cell free system, or in vivo, e.g., in a two-hybrid interaction trap assay. This method can be used to identify naturally occurring molecules which interact with subject MKP polypeptide. It can also be used to find natural or synthetic inhibitors of subject MKP polypeptide. Screening methods are discussed in more detail below.

Screening Assays

The invention provides methods (also referred to herein as “screening assays”) for identifying modulators, i.e., candidate or test compounds or agents (e.g., proteins, peptides, peptidomimetics, peptoids, small molecules or other drugs) which bind to MKP proteins, have a stimulatory or inhibitory effect on, for example, MKP expression or MKP activity, or have a stimulatory or inhibitory effect on, for example, the expression or activity of a MKP substrate. Compounds thus identified can be used to modulate the activity of target gene products (e.g., MKP genes) in a therapeutic protocol, to elaborate the biological function of the target gene product, or to identify compounds that disrupt normal target gene interactions.

In one embodiment, the invention provides assays for screening candidate or test compounds which are substrates of a MKP protein or polypeptide or a biologically active portion thereof. In another embodiment, the invention provides assays for screening candidate or test compounds which bind to or modulate the activity of a MKP protein or polypeptide or a biologically active portion thereof.

The test compounds of the invention can be obtained using any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann et al. (1994) J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909-13; Erb et al. (1994) Proc. Natl. Acad. Sci. USA 91:11422-426; Zuckermann et al. (1994). J. Med. Chem. 37:2678-85; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233-51.

Libraries of compounds can be presented in solution (e.g., Houghten (1992) Biotechniques 13:412-421), or on beads (Lam (1991) Nature 354:82-84), chips (Fodor (1993) Nature 364:555-556), bacteria (Ladner, U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.).

In one embodiment, an assay is a cell-based assay in which a cell which expresses a MKP protein or biologically active portion thereof is contacted with a test compound, and the ability of the test compound to modulate MKP activity is determined. Determining the ability of the test compound to modulate MKP activity can be accomplished by monitoring, for example, phosphatase activity, or the ability to dephosphorylate a substrate, or the ability to modulate (e.g., inactivate, e.g., via dephosphorylation) enzymes involved in signaling pathways, e.g., MAP kinases. The cell, for example, can be of mammalian origin, e.g., human.

The ability of the test compound to modulate MKP binding to a compound, e.g., a MKP substrate, or to bind to MKP can also be evaluated. This can be accomplished, for example, by coupling the compound, e.g., the substrate, with a radioisotope or enzymatic label such that binding of the compound, e.g., the substrate, to MKP can be determined by detecting the labeled compound, e.g., substrate, in a complex. Alternatively, MKP could be coupled with a radioisotope or enzymatic label to monitor the ability of a test compound to modulate MKP binding to a MKP substrate in a complex. For example, compounds (e.g., MKP substrates) can be labeled with ¹²⁵I, ¹⁴C, ³⁵S or ³H., either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

The ability of a compound (e.g., a MKP substrate) to interact with MKP with or without the labeling of any of the interactants can be evaluated. For example, a microphysiometer can be used to detect the interaction of a compound with MKP without the labeling of either the compound or the MKP. McConnell et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and MKP.

In yet another embodiment, a cell-free assay is provided in which a MKP protein or biologically active portion thereof is contacted with a test compound and the ability of the test compound to bind to the MKP protein or biologically active portion thereof is evaluated. Preferred biologically active portions of the MKP proteins to be used in assays of the invention include fragments which participate in interactions with non-MKP molecules, e.g., fragments with high surface probability scores.

Soluble and/or membrane-bound forms of isolated proteins (e.g., MKP proteins or biologically active portions thereof) can be used in the cell-free assays of the invention. When membrane-bound forms of the protein are used, it may be desirable to utilize a solubilizing agent. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)_{n,} 3-[(3-cholamidopropyl)dimethylamino]-1-propane sulfonate (CHAPS), 3-[(3-cholamidopropyl)dimethylamminio]-2-hydroxy- 1-propane sulfonate (CHAPSO), or N-dodecyl=N,N-dimethyl-3-ammonio- 1-propane sulfonate.

Cell-free assays involve preparing a reaction mixture of the target gene protein and the test compound under conditions and for a time sufficient to allow the two components to interact and bind, thus forming a complex that can be removed and/or detected.

The interaction between two molecules can also be detected, e.g., using fluorescence energy transfer (FET) (see, for example, Lakowicz et al., U.S. Pat. No. 5,631,169; Stavrianopoulos, et al., U.S. Pat. No. 4,868,103). A fluorophore label on the first, ‘donor’ molecule is selected such that its emitted fluorescent energy will be absorbed by a fluorescent label on a second, ‘acceptor’ molecule, which in turn is able to fluoresce due to the absorbed energy. Alternately, the ‘donor’ protein molecule can simply utilize the natural fluorescent energy of tryptophan residues. Labels are chosen that emit different wavelengths of light, such that the ‘acceptor’ molecule label can be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determining the ability of the MKP protein to bind to a target molecule can be accomplished using real-time Biomolecular Interaction Analysis (BIA) (see, e.g., Sjolander and Urbaniczky (1991) Anal. Chem. 63:2338-2345 and Szabo et al. (1995) Curr. Opin. Struct. Biol. 5:699-705). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

In one embodiment, the target gene product or the test substance is anchored onto a solid phase. The target gene product/test compound complexes anchored on the solid phase can be detected at the end of the reaction. Preferably, the target gene product can be anchored onto a solid surface, and the test compound, (which is not anchored), can be labeled, either directly or indirectly, with detectable labels discussed herein.

It may be desirable to immobilize either MKP, an anti-MKP antibody or its target molecule to facilitate separation of complexed from uncomplexed forms of one or both of the proteins, as well as to accommodate automation of the assay. Binding of a test compound to a MKP protein, or interaction of a MKP protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase/MKP fusion proteins or glutathione-S-transferase/target fusion proteins can be adsorbed onto glutathione sepharose beads (Sigma Chemical, St. Louis, Mo.) or glutathione derivatized microtiter plates, which are then combined with the test compound or the test compound and either the non-adsorbed target protein or MKP protein, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of MKP binding or activity determined using standard techniques.

Other techniques for immobilizing either a MKP protein or a target molecule on matrices include using conjugation of biotin and streptavidin. Biotinylated MKP protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific or selective for the immobilized component (the antibody, in turn, can be directly labeled or indirectly labeled with, e.g., a labeled anti-Ig antibody).

In one embodiment, this assay is performed utilizing antibodies reactive with MKP protein or target molecules but which do not interfere with binding of the MKP protein to its target molecule. Such antibodies can be derivatized to the wells of the plate, and unbound target or MKP protein trapped in the wells by antibody conjugation. Methods for detecting such complexes, in addition to those described above for the GST-immobilized complexes, include immunodetection of complexes using antibodies reactive with the MKP protein or target molecule, as well as enzyme-linked assays which rely on detecting an enzymatic activity associated with the MKP protein or target molecule.

Alternatively, cell free assays can be conducted in a liquid phase. In such an assay, the reaction products are separated from unreacted components, by any of a number of standard techniques, including but not limited to: differential centrifugation (see, for example, Rivas and Minton (1993) Trends Biochem Sci 18:284-7); chromatography (gel filtration chromatography, ion-exchange chromatography); electrophoresis (see, e.g., Ausubel et al., eds. (1999) Current Protocols in Molecular Biology, J. Wiley, New York.); and immunoprecipitation (see, for example, Ausubel et al., eds. (1999) Current Protocols in Molecular Biology, J. Wiley, New York). Such resins and chromatographic techniques are known to one skilled in the art (see, e.g., Heegaard (1998) J Mol Recognit 11: 141-8; Hage and Tweed (1997) J Chromatogr B Biomed Sci Appl. 699:499-525). Further, fluorescence energy transfer can also be conveniently utilized, as described herein, to detect binding without further purification of the complex from solution.

In a preferred embodiment, the assay includes contacting the MKP protein or biologically active portion thereof with a known compound which binds MKP to form an assay mixture, contacting the assay mixture with a test compound, and determining the ability of the test compound to interact with a MKP protein, wherein determining the ability of the test compound to interact with a MKP protein includes determining the ability of the test compound to preferentially bind to MKP or biologically active portion thereof, or to modulate the activity of a target molecule, as compared to the known compound.

The target gene products of the invention can, in vivo, interact with one or more cellular or extracellular macromolecules, such as proteins. For the purposes of this discussion, such cellular and extracellular macromolecules are referred to herein as “binding partners.” Compounds that disrupt such interactions can be useful in regulating the activity of the target gene product. Such compounds can include, but are not limited to molecules such as antibodies, peptides, and small molecules. The preferred target genes/products for use in this embodiment are the MKP genes herein identified. In an alternative embodiment, the invention provides methods for determining the ability of the test compound to modulate the activity of a MKP protein through modulation of the activity of a downstream effector of a MKP target molecule. For example, the activity of the effector molecule on an appropriate target can be determined, or the binding of the effector to an appropriate target can be determined, as previously described.

To identify compounds that interfere with the interaction between the target gene product and its cellular or extracellular binding partner(s), a reaction mixture containing the target gene product and the binding partner is prepared, under conditions and for a time sufficient, to allow the two products to form complex. In order to test an inhibitory agent, the reaction mixture is provided in the presence and absence of the test compound. The test compound can be initially included in the reaction mixture, or can be added at a time subsequent to the addition of the target gene and its cellular or extracellular binding partner. Control reaction mixtures are incubated without the test compound or with a placebo. The formation of any complexes between the target gene product and the cellular or extracellular binding partner is then detected. The formation of a complex in the control reaction, but not in the reaction mixture containing the test compound, indicates that the compound interferes with the interaction of the target gene product and the interactive binding partner. Additionally, complex formation within reaction mixtures containing the test compound and normal target gene product can also be compared to complex formation within reaction mixtures containing the test compound and mutant target gene product. This comparison can be important in those cases wherein it is desirable to identify compounds that disrupt interactions of mutant but not normal target gene products.

These assays can be conducted in a heterogeneous or homogeneous format. Heterogeneous assays involve anchoring either the target gene product or the binding partner onto a solid phase, and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the target gene products and the binding partners, e.g., by competition, can be identified by conducting the reaction in the presence of the test substance. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the target gene product or the interactive cellular or extracellular binding partner, is anchored onto a solid surface (e.g., a microtiter plate), while the non-anchored species is labeled, either directly or indirectly. The anchored species can be immobilized by non-covalent or covalent attachments. Alternatively, an immobilized antibody specific or selective for the species to be anchored can be used to anchor the species to the solid surface.

In order to conduct the assay, the partner of the immobilized species is exposed to the coated surface with or without the test compound. After the reaction is complete, unreacted components are removed (e.g., by washing) and any complexes formed will remain immobilized on the solid surface. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific or selective for the initially non-immobilized species (the antibody, in turn, can be directly labeled or indirectly labeled with, e.g., a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from unreacted components, and complexes detected; e.g., using an immobilized antibody specific or selective for one of the binding components to anchor any complexes formed in solution, and a labeled antibody specific or selective for the other partner to detect anchored complexes. Again, depending upon the order of addition of reactants to the liquid phase, test compounds that inhibit complex or that disrupt preformed complexes can be identified.

In an alternate embodiment of the invention, a homogeneous assay can be used. For example, a preformed complex of the target gene product and the interactive cellular or extracellular binding partner product is prepared in that either the target gene products or their binding partners are labeled, but the signal generated by the label is quenched due to complex formation (see, e.g., U.S. Pat. No. 4,109,496 that utilizes this approach for immunoassays). The addition of a test substance that competes with and displaces one of the species from the preformed complex will result in the generation of a signal above background. In this way, test substances that disrupt target gene product-binding partner interaction can be identified.

In yet another aspect, the MKP proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8: 1693-1696; and Brent WO94/10300), to identify other proteins, which bind to or interact with MKP (“MKP-binding proteins” or “MKP-bp”) and are involved in MKP activity. Such MKP-bps can be activators or inhibitors of signals by the MKP proteins or MKP targets as, for example, downstream elements of a MKP-mediated signaling pathway.

The two-hybrid system is based on the modular nature of most transcription factors, which consist of separable DNA-binding and activation domains. Briefly, the assay utilizes two different DNA constructs. In one construct, the gene that codes for a MKP protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. (Alternatively the: MKP protein can be the fused to the activator domain.) If the “bait” and the “prey” proteins are able to interact, in vivo, forming a MKP-dependent complex, the DNA-binding and activation domains of the transcription factor are brought into close proximity. This proximity allows transcription of a reporter gene (e.g., lacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the MKP protein.

In another embodiment, modulators of MKP expression are identified. For example, a cell or cell free mixture is contacted with a candidate compound and the expression of MKP mRNA or protein evaluated relative to the level of expression of MKP mRNA or protein in the absence of the candidate compound. When expression of MKP mRNA or protein is greater in the presence of the candidate compound than in its absence, the candidate compound is identified as a stimulator of MKP mRNA or protein expression. Alternatively, when expression of MKP mRNA or protein is less (statistically significantly less) in the presence of the candidate compound than in its absence, the candidate compound is identified as an inhibitor of MKP mRNA or protein expression. The level of MKP mRNA or protein expression can be determined by methods described herein for detecting MKP mRNA or protein.

In another aspect, the invention pertains to a combination of two or more of the assays described herein. For example, a modulating agent can be identified using a cell-based or a cell free assay, and the ability of the agent to modulate the activity of a MKP protein can be confirmed in vivo, e.g., in an animal such as a mouse model of obesity (e.g., an ob/ob, A^{Y}, or db/db mouse).

This invention further pertains to novel agents identified by the above-described screening assays. Accordingly, it is within the scope of this invention to further use an agent identified as described herein (e.g., a MKP modulating agent, an antisense MKP nucleic acid molecule, a MKP-specific antibody, or a MKP-binding partner) in an appropriate animal model to determine the efficacy, toxicity, side effects, or mechanism of action, of treatment with such an agent. Furthermore, novel agents identified by the above-described screening assays can be used for treatments as described herein.

Detection Assays

Portions or fragments of the nucleic acid sequences identified herein can be used as polynucleotide reagents. For example, these sequences can be used to: (i) map their respective genes on a chromosome e.g., to locate gene regions associated with genetic disease or to associate MKP with a disease; (ii) identify an individual from a minute biological sample (tissue typing); and (iii) aid in forensic identification of a biological sample. These applications are described in the subsections below.

Chromosome Mapping

The MKP nucleotide sequences or portions thereof can be used to map the location of the MKP genes on a chromosome. This process is called chromosome mapping. Chromosome mapping is useful in correlating the MKP sequences with genes associated with disease.

Briefly, MKP genes can be mapped to chromosomes by preparing PCR primers (preferably 15-25 bp in length) from the MKP nucleotide sequences. These primers can then be used for PCR screening of somatic cell hybrids containing individual human chromosomes. Only those hybrids containing the human gene corresponding to the MKP sequences will yield an amplified fragment.

A panel of somatic cell hybrids in which each cell line contains either a single human chromosome or a small number of human chromosomes, and a full set of mouse chromosomes, can allow easy mapping of individual genes to specific human chromosomes. (D'Eustachio et al. (1983) Science 220:919-924).

Other mapping strategies e.g., in situ hybridization (described in Fan et al. (1990) Proc. Natl. Acad. Sci. USA, 87:6223-27), pre-screening with labeled flow-sorted chromosomes, and pre-selection by hybridization to chromosome specific cDNA libraries can be used to map MKP to a chromosomal location.

Fluorescence in situ hybridization (FISH) of a DNA sequence to a metaphase chromosomal spread can further be used to provide a precise chromosomal location in one step. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of this technique, see Verma et al. (1988) Human Chromosomes: A Manual of Basic Techniques, Pergamon Press, New York).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in McKusick, Mendelian Inheritance in Man, available on-line through Johns Hopkins University Welch Medical Library). The relationship between a gene and a disease, mapped to the same chromosomal region, can then be identified through linkage analysis (co-inheritance of physically adjacent genes), described in, for example, Egeland et al. (1987) Nature, 325:783-787.

Moreover, differences in the DNA sequences between individuals affected and unaffected with a disease associated with the MKP gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

MKP sequences can be used to identify individuals from biological samples using, e.g., restriction fragment length polymorphism (RFLP). In this technique, an individual's genomic DNA is digested with one or more restriction enzymes, the fragments separated, e.g., in a Southern blot, and probed to yield bands for identification. The sequences of the invention are useful as additional DNA markers for RFLP (described in U.S. Pat. No. 5,272,057).

Furthermore, the sequences of the invention can also be used to determine the actual base-by-base DNA sequence of selected portions of an individual's genome. Thus, the MKP nucleotide sequences described herein can be used to prepare two PCR primers from the 5′ and 3′ ends of the sequences. These primers can then be used to amplify an individual's DNA and subsequently sequence it. Panels of corresponding DNA sequences from individuals, prepared in this manner, can provide unique individual identifications, as each individual will have a unique set of such DNA sequences due to allelic differences.

Allelic variation occurs to some degree in the coding regions of these sequences, and to a greater degree in the noncoding regions. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NO:1 can provide positive individual identification with a panel of perhaps 10 to 1,000 primers which each yield a noncoding amplified sequence of 100 bases. If predicted coding sequences, such as those in SEQ ID NO:3 are used, a more appropriate number of primers for positive individual identification would be 500-2,000.

If a panel of reagents from MKP nucleotide sequences described herein is used to generate a unique identification database for an individual, those same reagents can later be used to identify tissue from that individual. Using the unique identification database, positive identification of the individual, living or dead, can be made from extremely small tissue samples.

Use of Partial MKP Sequences in Forensic Biology

DNA-based identification techniques can also be used in forensic biology. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions of SEQ ID NO:1 (e.g., fragments derived from the noncoding regions of SEQ ID NO:1 having a length of at least 20 bases, preferably at least 30 bases) are particularly appropriate for this use.

The MKP nucleotide sequences described herein can further be used to provide polynucleotide reagents, e.g., labeled or labelable probes which can be used in, for example, an in situ hybridization technique, to identify a specific tissue. This can be very useful in cases where a forensic pathologist is presented with a tissue of unknown origin. Panels of such MKP probes can be used to identify tissue by species and/or by organ type.

In a similar fashion, these reagents, e.g., MKP primers or probes can be used to screen tissue culture for contamination (i.e. screen for the presence of a mixture of different types of cells in a culture).

Predictive Medicine

The invention also pertains to the field of predictive medicine in which diagnostic assays, prognostic assays, and monitoring clinical trials are used for prognostic (predictive) purposes to thereby treat an individual.

Generally, the invention provides, a method of determining if a subject is at risk for a disorder related to a lesion in or the misexpression of a gene which encodes MKP.

Such disorders include, e.g., a disorder associated with the misexpression of MKP gene, e.g., a metabolic disorder.

The method includes one or more of the following:

detecting, in a tissue of the subject, the presence or absence of a mutation which affects the expression of the MKP gene, or detecting the presence or absence of a mutation in a region which controls the expression of the gene, e.g., a mutation in the 5′ control region;

detecting, in a tissue of the subject, the presence or absence of a mutation which alters the structure of the MKP gene;

detecting, in a tissue of the subject, the misexpression of the MKP gene, at the mRNA level, e.g., detecting a non-wild type level of an mRNA;

detecting, in a tissue of the subject, the misexpression of the gene, at the protein level, e.g., detecting a non-wild type level of a MKP polypeptide.

In preferred embodiments the method includes: ascertaining the existence of at least one of: a deletion of one or more nucleotides from the MKP gene; an insertion of one or more nucleotides into the gene, a point mutation, e.g., a substitution of one or more nucleotides of the gene, a gross chromosomal rearrangement of the gene, e.g., a translocation, inversion, or deletion.

For example, detecting the genetic lesion can include: (i) providing a probe/primer including an oligonucleotide containing a region of nucleotide sequence which hybridizes to a sense or antisense sequence from SEQ ID NO:1, or naturally occurring mutants thereof or 5′ or 3′ flanking sequences naturally associated with the MKP gene; (ii) exposing the probe/primer to nucleic acid of the tissue; and detecting, by hybridization, e.g., in situ hybridization, of the probe/primer to the nucleic acid, the presence or absence of the genetic lesion.

In preferred embodiments detecting the misexpression includes ascertaining the existence of at least one of: an alteration in the level of a messenger RNA transcript of the MKP gene; the presence of a non-wild type splicing pattern of a messenger RNA transcript of the gene; or a non-wild type level of MKP.

Methods of the invention can be used prenatally or to determine if a subject's offspring will be at risk for a disorder.

In preferred embodiments the method includes determining the structure of a MKP gene, an abnormal structure being indicative of risk for the disorder.

In preferred embodiments the method includes contacting a sample from the subject with an antibody to the MKP protein or a nucleic acid, which hybridizes specifically with the gene. These and other embodiments are discussed below.

Diagnostic and Prognostic Assays

The presence, level, or absence of MKP protein or nucleic acid in a biological sample can be evaluated by obtaining a biological sample from a test subject and contacting the biological sample with a compound or an agent capable of detecting MKP protein or nucleic acid (e.g., mRNA, genomic DNA) that encodes MKP protein such that the presence of MKP protein or nucleic acid is detected in the biological sample. The term “biological sample” includes tissues, cells and biological fluids isolated from a subject, as well as tissues, cells and fluids present within a subject. A preferred biological sample is serum. The level of expression of the MKP gene can be measured in a number of ways, including, but not limited to: measuring the mRNA encoded by the MKP genes; measuring the amount of protein encoded by the MKP genes; or measuring the activity of the protein encoded by the MKP genes.

The level of mRNA corresponding to the MKP gene in a cell can be determined both by in situ and by in vitro formats.

The isolated mRNA can be used in hybridization or amplification assays that include, but are not limited to, Southern or Northern analyses, polymerase chain reaction analyses and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, for example, a full-length MKP nucleic acid, such as the nucleic acid of SEQ ID NO:1, or a portion thereof, such as an oligonucleotide of at least 7, 15, 30, 50, 100, 250 or 500 nucleotides in length and sufficient to specifically hybridize under stringent conditions to MKP mRNA or genomic DNA. Other suitable probes for use in the diagnostic assays are described herein.

In one format, mRNA (or cDNA) is immobilized on a surface and contacted with the probes, for example by running the isolated mRNA on an agarose gel and transferring the mRNA from the gel to a membrane, such as nitrocellulose. In an alternative format, the probes are immobilized on a surface and the mRNA (or cDNA) is contacted with the probes, for example, in a two-dimensional gene chip array. A skilled artisan can adapt known mRNA detection methods for use in detecting the level of mRNA encoded by the MKP genes.

The level of mRNA in a sample that is encoded by one of MKP can be evaluated with nucleic acid amplification, e.g., by rtPCR (Mullis (1987) U.S. Pat. No. 4,683,202), ligase chain reaction (Barany (1991) Proc. Natl. Acad. Sci. USA 88:189-193), self sustained sequence replication (Guatelli et al., (1990) Proc. Natl. Acad. Sci. USA 87:1874-1878), transcriptional amplification system (Kwoh et al., (1989), Proc. Natl. Acad. Sci. USA 86:1173-1177), Q-Beta Replicase (Lizardi et al., (1988) Bio/Technology 6:1197), rolling circle replication (Lizardi et al., U.S. Pat. No. 5,854,033) or any other nucleic acid amplification method, followed by the detection of the amplified molecules using techniques known in the art. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, a cell or tissue sample can be prepared/processed and immobilized on a support, typically a glass slide, and then contacted with a probe that can hybridize to mRNA that encodes the MKP gene being analyzed.

In another embodiment, the methods further contacting a control sample with a compound or agent capable of detecting MKP mRNA, or genomic DNA, and comparing the presence of MKP mRNA or genomic DNA in the control sample with the presence of MKP mRNA or genomic DNA in the test sample.

A variety of methods can be used to determine the level of protein encoded by MKP. In general, these methods include contacting an agent that selectively binds to the protein, such as an antibody with a sample, to evaluate the level of protein in the sample. In a preferred embodiment, the antibody bears a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with a detectable substance. Examples of detectable substances are provided herein.

The detection methods can be used to detect MKP protein in a biological sample in vitro as well as in vivo. In vitro techniques for detection of MKP protein include enzyme linked immunosorbent assays (ELISAs), immunoprecipitations, immunofluorescence, enzyme immunoassay (EIA), radioimmunoassay (RIA), and Western blot analysis. In vivo techniques for detection of MKP protein include introducing into a subject a labeled anti-MKP antibody. For example, the antibody can be labeled with a radioactive marker whose presence and location in a subject can be detected by standard imaging techniques.

In another embodiment, the methods further include contacting the control sample with a compound or agent capable of detecting MKP protein, and comparing the presence of MKP protein in the control sample with the presence of MKP protein in the test sample.

The invention also includes kits for detecting the presence of MKP in a biological sample. For example, the kit can include a compound or agent capable of detecting MKP protein or mRNA in a biological sample; and a standard. The compound or agent can be packaged in a suitable container. The kit can further comprise instructions for using the kit to detect MKP protein or nucleic acid.

For antibody-based kits, the kit can include: (1) a first antibody (e.g., attached to a solid support) which binds to a polypeptide corresponding to a marker of the invention; and, optionally, (2) a second, different antibody which binds to either the polypeptide or the first antibody and is conjugated to a detectable agent.

For oligonucleotide-based kits, the kit can include: (1) an oligonucleotide, e.g., a detectably labeled oligonucleotide, which hybridizes to a nucleic acid sequence encoding a polypeptide corresponding to a marker of the invention or (2) a pair of primers useful for amplifying a nucleic acid molecule corresponding to a marker of the invention. The kit can also includes a buffering agent, a preservative, or a protein stabilizing agent. The kit can also includes components necessary for detecting the detectable agent (e.g., an enzyme or a substrate). The kit can also contain a control sample or a series of control samples which can be assayed and compared to the test sample contained. Each component of the kit can be enclosed within an individual container and all of the various containers can be within a single package, along with instructions for interpreting the results of the assays performed using the kit.

The diagnostic methods described herein can identify subjects having, or at risk of developing, a disease or disorder associated with misexpressed or aberrant or unwanted MKP expression or activity. As used herein, the term “unwanted” includes an unwanted phenomenon involved in a biological response such as pain or deregulated cell proliferation.

In one embodiment, a disease or disorder associated with aberrant or unwanted MKP expression or activity is identified. A test sample is obtained from a subject and MKP protein or nucleic acid (e.g., mRNA or genomic DNA) is evaluated, wherein the level, e.g., the presence or absence, of MKP protein or nucleic acid is diagnostic for a subject having or at risk of developing a disease or disorder associated with aberrant or unwanted MKP expression or activity. As used herein, a “test sample” refers to a biological sample obtained from a subject of interest, including a biological fluid (e.g., serum), cell sample, or tissue.

The prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant or unwanted MKP expression or activity. For example, such methods can be used to determine whether a subject can be effectively treated with an agent for a metabolic disorder (e.g., obesity).

The methods of the invention can also be used to detect genetic alterations in a MKP gene, thereby determining if a subject with the altered gene is at risk for a disorder characterized by misregulation in MKP protein activity or nucleic acid expression, such as a metabolic disorder (e.g., obesity). In preferred embodiments, the methods include detecting, in a sample from the subject, the presence or absence of a genetic alteration characterized by at least one of an alteration affecting the integrity of a gene encoding a MKP-protein, or the mis-expression of the MKP gene. For example, such genetic alterations can be detected by ascertaining the existence of at least one of 1) a deletion of one or more nucleotides from a MKP gene; 2) an addition of one or more nucleotides to a MKP gene; 3) a substitution of one or more nucleotides of a MKP gene, 4) a chromosomal rearrangement of a MKP gene; 5) an alteration in the level of a messenger RNA transcript of a MKP gene, 6) aberrant modification of a MKP gene, such as of the methylation pattern of the genomic DNA, 7) the presence of a non-wild type splicing pattern of a messenger RNA transcript of a MKP gene, 8) a non-wild type level of a MKP-protein, 9) allelic loss of a MKP gene, and 10) inappropriate post-translational modification of a MKP-protein.

An alteration can be detected without a probe/primer in a polymerase chain reaction, such as anchor PCR or RACE PCR, or, alternatively, in a ligation chain reaction (LCR), the latter of which can be particularly useful for detecting point mutations in the MKP-gene. This method can include the steps of collecting a sample of cells from a subject, isolating nucleic acid (e.g., genomic, mRNA or both) from the sample, contacting the nucleic acid sample with one or more primers which specifically hybridize to a MKP gene under conditions such that hybridization and amplification of the MKP gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein. Alternatively, other amplification methods described herein or known in the art can be used.

In another embodiment, mutations in a MKP gene from a sample cell can be identified by detecting alterations in restriction enzyme cleavage patterns. For example, sample and control DNA is isolated, amplified (optionally), digested with one or more restriction endonucleases, and fragment length sizes are determined, e.g., by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in MKP can be identified by hybridizing a sample and control nucleic acids, e.g., DNA or RNA, two dimensional arrays, e.g., chip based arrays. Such arrays include a plurality of addresses, each of which is positionally distinguishable from the other. A different probe is located at each address of the plurality. The arrays can have a high density of addresses, e.g., can contain hundreds or thousands of oligonucleotides probes (Cronin et al. (1996) Human Mutation 7: 244-255; Kozal et al. (1996) Nature Medicine 2: 753-759). For example, genetic mutations in MKP can be identified in two dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the MKP gene and detect mutations by comparing the sequence of the sample MKP with the corresponding wild-type (control) sequence. Automated sequencing procedures can be utilized when performing the diagnostic assays (Naeve et al. (1995) Biotechniques 19:448-53), including sequencing by mass spectrometry.

Other methods for detecting mutations in the MKP gene include methods in which protection from cleavage agents is used to detect mismatched bases in RNA/RNA or RNA/DNA heteroduplexes (Myers et al. (1985) Science 230:1242; Cotton et al. (1988) Proc. Natl Acad Sci USA 85:4397; Saleeba et al. (1992) Methods Enzymol. 217:286-295).

In still another embodiment, the mismatch cleavage reaction employs one or more proteins that recognize mismatched base pairs in double-stranded DNA (so called “DNA mismatch repair” enzymes) in defined systems for detecting and mapping point mutations in MKP cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662; U.S. Pat. No. 5,459,039).

In other embodiments, alterations in electrophoretic mobility will be used to identify mutations in MKP genes. For example, single strand conformation polymorphism (SSCP) can be used to detect differences in electrophoretic mobility between mutant and wild type nucleic acids (Orita et al. (1989) Proc Natl. Acad. Sci USA: 86:2766, see also Cotton (1993) Mutat. Res. 285:125-144; and Hayashi (1992) Genet. Anal. Tech. Appl. 9:73-79). Single-stranded DNA fragments of sample and control MKP nucleic acids will be denatured and allowed to renature. The secondary structure of single-stranded nucleic acids varies according to sequence, the resulting alteration in electrophoretic mobility enables the detection of even a single base change. The DNA fragments can be labeled or detected with labeled probes. The sensitivity of the assay can be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment, the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension (Saiki et al. (1986) Nature 324:163); Saiki et al. (1989) Proc. Natl Acad. Sci USA 86:6230).

Alternatively, allele specific amplification technology which depends on selective PCR amplification can be used in conjunction with the instant invention. Oligonucleotides used as primers for specific amplification can carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification can also be performed using Taq ligase for amplification (Barany (1991) Proc. Natl. Acad. Sci USA 88:189-93). In such cases, ligation will occur only if there is a perfect match at the 3′ end of the 5′ sequence making it possible to detect the presence of a known mutation at a specific site by looking for the presence or absence of amplification.

The methods described herein can be performed, for example, by utilizing pre-packaged diagnostic kits comprising at least one probe nucleic acid or antibody reagent described herein, which can be conveniently used, e.g., in clinical settings to diagnose patients exhibiting symptoms or family history of a disease or illness involving a MKP gene.

Use of MKP Molecules as Surrogate Markers

The MKP molecules of the invention are also useful as markers of disorders or disease states, as markers for precursors of disease states, as markers for predisposition of disease states, as markers of drug activity, or as markers of the pharmacogenomic profile of a subject. Using the methods described herein, the presence, absence and/or quantity of the MKP molecules of the invention can be detected, and can be correlated with one or more biological states in vivo. For example, the MKP molecules of the invention can serve as surrogate markers for one or more disorders or disease states or for conditions leading up to disease states. As used herein, a “surrogate marker” is an objective biochemical marker which correlates with the absence or presence of a disease or disorder, or with the progression of a disease or disorder (e.g., with the presence or absence of a tumor). The presence or quantity of such markers is independent of the disease. Therefore, these markers can serve to indicate whether a particular course of treatment is effective in lessening a disease state or disorder. Surrogate markers are of particular use when the presence or extent of a disease state or disorder is difficult to assess through standard methodologies (e.g., early stage tumors), or when an assessment of disease progression is desired before a potentially dangerous clinical endpoint is reached (e.g., an assessment of cardiovascular disease can be made using cholesterol levels as a surrogate marker, and an analysis of HIV infection can be made using HIV RNA levels as a surrogate marker, well in advance of the undesirable clinical outcomes of myocardial infarction or fully-developed AIDS). Examples of the use of surrogate markers in the art include: Koomen et al. (2000) J. Mass. Spectrom. 35: 258-264; and James (1994) AIDS Treatment News Archive 209.

The MKP molecules of the invention are also useful as pharmacodynamic markers. As used herein, a “pharmacodynamic marker” is an objective biochemical marker which correlates specifically with drug effects. The presence or quantity of a pharmacodynamic marker is not related to the disease state or disorder for which the drug is being administered; therefore, the presence or quantity of the marker is indicative of the presence or activity of the drug in a subject. For example, a pharmacodynamic marker can be indicative of the concentration of the drug in a biological tissue, in that the marker is either expressed or transcribed or not expressed or transcribed in that tissue in relationship to the level of the drug. In this fashion, the distribution or uptake of the drug can be monitored by the pharmacodynamic marker. Similarly, the presence or quantity of the pharmacodynamic marker can be related to the presence or quantity of the metabolic product of a drug, such that the presence or quantity of the marker is indicative of the relative breakdown rate of the drug in vivo. Pharmacodynamic markers are of particular use in increasing the sensitivity of detection of drug effects, particularly when the drug is administered in low doses. Since even a small amount of a drug can be sufficient to activate multiple rounds of marker (e.g., a MKP marker) transcription or expression, the amplified marker can be in a quantity which is more readily detectable than the drug itself. Also, the marker can be more easily detected due to the nature of the marker itself; for example, using the methods described herein, anti-MKP antibodies can be employed in an immune-based detection system for a MKP protein marker, or MKP-specific radiolabeled probes can be used to detect a MKP mRNA marker. Furthermore, the use of a pharmacodynamic marker can offer mechanism-based prediction of risk due to drug treatment beyond the range of possible direct observations. Examples of the use of pharmacodynamic markers in the art include: Matsuda et al. U.S. Pat. No. 6,033,862; Hattis et al. (1991) Env. Health Perspect. 90: 229-238; Schentag (1999) Am. J. Health-Syst. Pharm. 56 Suppl. 3: S21-S24; and Nicolau (1999) Am. J. Health-Syst. Pharm. 56 Suppl. 3: S16-S20.

The MKP molecules of the invention are also useful as pharmacogenomic markers. As used herein, a “pharmacogenomic marker” is an objective biochemical marker which correlates with a specific clinical drug response or susceptibility in a subject (see, e.g., McLeod et al. (1999) Eur. J. Cancer 35:1650-1652). The presence or quantity of the pharmacogenomic marker is related to the predicted response of the subject to a specific drug or class of drugs prior to administration of the drug. By assessing the presence or quantity of one or more pharmacogenomic markers in a subject, a drug therapy which is most appropriate for the subject, or which is predicted to have a greater degree of success, can be selected. For example, based on the presence or quantity of RNA, or protein (e.g., MKP protein or RNA) for specific tumor markers in a subject, a drug or course of treatment can be selected that is optimized for the treatment of the specific tumor likely to be present in the subject. Similarly, the presence or absence of a specific sequence mutation in MKP DNA can correlate with a MKP drug response. The use of pharmacogenomic markers therefore permits the application of the most appropriate treatment for each subject without having to administer the therapy.

Pharmaceutical Compositions

The nucleic acid and polypeptides, fragments thereof, as well as anti-MKP antibodies (also referred to herein as “active compounds”) of the invention can be incorporated into pharmaceutical compositions. Such compositions typically include the nucleic acid molecule, protein, or antibody and a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit high therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The protein or polypeptide can be administered one time per week for between about 1 to 10 weeks, preferably between 2 to 8 weeks, more preferably between about 3 to 7 weeks, and even more preferably for about 4, 5, or 6 weeks. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody, unconjugated or conjugated as described herein, can include a single treatment or, preferably, can include a series of treatments.

For antibodies, the preferred dosage is 0.1 mg/kg of body weight (generally 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al. ((1997) J. Acquired Immune Deficiency Syndromes and Human Retrovirology 14:193).

The invention encompasses agents which modulate expression or activity. An agent can, for example, be a small molecule. For example, such small molecules include, but are not limited to, peptides, peptidomimetics (e.g., peptoids), amino acids, amino acid analogs, polynucleotides, polynucleotide analogs, nucleotides, nucleotide analogs, organic or inorganic compounds (i.e.,. including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. When one or more of these small molecules is to be administered to an animal (e.g., a human) in order to modulate expression or activity of a polypeptide or nucleic acid of the invention, a physician, veterinarian, or researcher can, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

Methods of Treatment

The invention provides for both prophylactic and therapeutic methods of treating a subject at risk of (or susceptible to) a disorder or having a disorder associated with aberrant or unwanted MKP expression or activity. As used herein, the term “treatment” is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease. A therapeutic agent includes, but is not limited to, small molecules, peptides, antibodies, ribozymes and antisense oligonucleotides.

With regards to both prophylactic and therapeutic methods of treatment, such treatments can be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”.) Thus, another aspect of the invention provides methods for tailoring an individual's prophylactic or therapeutic treatment with either the MKP molecules of the invention or MKP modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

In one aspect, the invention provides a method for preventing in a subject, a disease or condition associated with an aberrant or unwanted MKP expression or activity, by administering to the subject a MKP or an agent which modulates MKP expression or at least one MKP activity. Subjects at risk for a disease which is caused or contributed to by aberrant or unwanted MKP expression or activity can be identified by, for example, any or a combination of diagnostic or prognostic assays as described herein. Administration of a prophylactic agent can occur prior to the manifestation of symptoms characteristic of the MKP aberrance, such that a disease or disorder is prevented or, alternatively, delayed in its progression. Depending on the type of MKP aberrance, for example, a MKP, MKP agonist or MKP antagonist agent can be used for treating the subject. The appropriate agent can be determined based on screening assays described herein.

It is possible that some MKP disorders can be caused, at least in part, by an abnormal level of gene product, or by the presence of a gene product exhibiting abnormal activity. As such, the reduction in the level and/or activity of such gene products would bring about the amelioration of disorder symptoms.

The MKP molecules can act as novel diagnostic targets and therapeutic agents for controlling one or more of metabolic disorders, endocrine disorders, and/or cellular growth, differentiative, or mitogenic (proliferative) disorders.

As discussed, successful treatment of MKP disorders can be brought about by techniques that serve to inhibit the expression or activity of target gene products. For example, compounds, e.g., an agent identified using an assays described above, that proves to exhibit negative modulatory activity, can be used in accordance with the invention to prevent and/or ameliorate symptoms of MKP disorders. Such molecules can include, but are not limited to peptides, phosphopeptides, small organic or inorganic molecules, or antibodies (including, for example, polyclonal, monoclonal, humanized, human, anti-idiotypic, chimeric or single chain antibodies, and Fab, F(ab′)₂ and Fab expression library fragments, scFV molecules, and epitope-binding fragments thereof).

Further, antisense and ribozyme molecules that inhibit expression of the target gene can also be used in accordance with the invention to reduce the level of target gene expression, thus effectively reducing the level of target gene activity. Still further, triple helix molecules can be utilized in reducing the level of target gene activity. Antisense, ribozyme and triple helix molecules are discussed above.

It is possible that the use of antisense, ribozyme, and/or triple helix molecules to reduce or inhibit mutant gene expression can also reduce or inhibit the transcription (triple helix) and/or translation (antisense, ribozyme) of mRNA produced by normal target gene alleles, such that the concentration of normal target gene product present can be lower than is necessary for a normal phenotype. In such cases, nucleic acid molecules that encode and express target gene polypeptides exhibiting normal target gene activity can be introduced into cells via gene therapy method. Alternatively, in instances in that the target gene encodes an extracellular protein, it can be preferable to co-administer normal target gene protein into the cell or tissue in order to maintain the requisite level of cellular or tissue target gene activity.

Another method by which nucleic acid molecules can be utilized in treating or preventing a disease characterized by MKP expression is through the use of aptamer molecules specific for MKP protein. Aptamers are nucleic acid molecules having a tertiary structure which permits them to specifically or selectively bind to protein ligands (see, e.g., Osborne et al. (1997) Curr. Opin. Chem Biol. 1: 5-9; and Patel (1997) Curr Opin Chem Biol 1:32-46). Since nucleic acid molecules can in many cases be more conveniently introduced into target cells than therapeutic protein molecules can be, aptamers offer a method by which MKP protein activity can be specifically decreased without the introduction of drugs or other molecules which can have pluripotent effects.

Antibodies can be generated that are both specific for target gene product and that reduce target gene product activity. Such antibodies can, therefore, by administered in instances whereby negative modulatory techniques are appropriate for the treatment of MKP disorders. For a description of antibodies, see the Antibody section above.

In circumstances wherein injection of an animal or a human subject with a MKP protein or epitope for stimulating antibody production is harmful to the subject, it is possible to generate an immune response against MKP through the use of anti-idiotypic antibodies (see, for example, Herlyn (1999) Ann Med 31:66-78; and Bhattacharya-Chatterjee and Foon (1998) Cancer Treat Res. 94:51-68). If an anti-idiotypic antibody is introduced into a mammal or human subject, it should stimulate the production of anti-anti-idiotypic antibodies, which should be specific to the MKP protein. Vaccines directed to a disease characterized by MKP expression can also be generated in this fashion.

In instances where the target antigen is intracellular and whole antibodies are used, internalizing antibodies can be preferred. Lipofectin or liposomes can be used to deliver the antibody or a fragment of the Fab region that binds to the target antigen into cells. Where fragments of the antibody are used, the smallest inhibitory fragment that binds to the target antigen is preferred. For example, peptides having an amino acid sequence corresponding to the Fv region of the antibody can be used. Alternatively, single chain neutralizing antibodies that bind to intracellular target antigens can also be administered. Such single chain antibodies can be administered, for example, by expressing nucleotide sequences encoding single-chain antibodies within the target cell population (see e.g., Marasco et al. (1993) Proc. Natl. Acad. Sci. USA 90:7889-7893).

The identified compounds that inhibit target gene expression, synthesis and/or activity can be administered to a patient at therapeutically effective doses to prevent, treat or ameliorate MKP disorders. A therapeutically effective dose refers to that amount of the compound sufficient to result in amelioration of symptoms of the disorders. Toxicity and therapeutic efficacy of such compounds can be determined by standard pharmaceutical procedures as described above.

The data obtained from the cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma can be measured, for example, by high performance liquid chromatography.

Another example of determination of effective dose for an individual is the ability to directly assay levels of “free” and “bound” compound in the serum of the test subject. Such assays can utilize antibody mimics and/or “biosensors” that have been created through molecular imprinting techniques. The compound which is able to modulate MKP activity is used as a template, or “imprinting molecule”, to spatially organize polymerizable monomers prior to their polymerization with catalytic reagents. The subsequent removal of the imprinted molecule leaves a polymer matrix which contains a repeated “negative image” of the compound and is able to selectively rebind the molecule under biological assay conditions. A detailed review of this technique can be seen in Ansell et al (1996) Current Opinion in Biotechnology 7:89-94 and in Shea (1994) Trends in Polymer Science 2:166-173. Such “imprinted” affinity matrixes are amenable to ligand-binding assays, whereby the immobilized monoclonal antibody component is replaced by an appropriately imprinted matrix. An example of the use of such matrixes in this way can be seen in Vlatakis et al (1993) Nature 361:645-647. Through the use of isotope-labeling, the “free” concentration of compound which modulates the expression or activity of MKP can be readily monitored and used in calculations of IC₅₀.

Such “imprinted” affinity matrixes can also be designed to include fluorescent groups whose photon-emitting properties measurably change upon local and selective binding of target compound. These changes can be readily assayed in real time using appropriate fiberoptic devices, in turn allowing the dose in a test subject to be quickly optimized based on its individual IC₅₀. An rudimentary example of such a “biosensor” is discussed in Kriz et al (1995) Analytical Chemistry 67:2142-2144.

Another aspect of the invention pertains to methods of modulating MKP expression or activity for therapeutic purposes. Accordingly, in an exemplary embodiment, the modulatory method of the invention involves contacting a cell with a MKP or agent that modulates one or more of the activities of MKP protein activity associated with the cell. An agent that modulates MKP protein activity can be an agent as described herein, such as a nucleic acid or a protein, a naturally-occurring target molecule of a MKP protein (e.g., a MKP substrate or receptor), a MKP antibody, a MKP agonist or antagonist, a peptidomimetic of a MKP agonist or antagonist, or other small molecule.

In one embodiment, the agent stimulates one or MKP activities. Examples of such stimulatory agents include active MKP protein and a nucleic acid molecule encoding MKP. In another embodiment, the agent inhibits one or more MKP activities. Examples of such inhibitory agents include antisense MKP nucleic acid molecules, anti-MKP antibodies, and MKP inhibitors. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant or unwanted expression or activity of a MKP protein or nucleic acid molecule. In one embodiment, the method involves administering an agent (e.g., an agent identified by a screening assay described herein), or combination of agents that modulates (e.g., up regulates or down regulates) MKP expression or activity. In another embodiment, the method involves administering a MKP protein or nucleic acid molecule as therapy to compensate for reduced, aberrant, or unwanted MKP expression or activity.

Stimulation of MKP activity is desirable in situations in which MKP is abnormally downregulated and/or in which increased MKP activity is likely to have a beneficial effect. For example, stimulation of MKP activity is desirable in situations in which a MKP is downregulated and/or in which increased MKP activity is likely to have a beneficial effect. Likewise, inhibition of MKP activity is desirable in situations in which MKP is abnormally upregulated and/or in which decreased MKP activity is likely to have a beneficial effect.

Pharmacogenomics

The MKP molecules of the invention, as well as agents, or modulators which have a stimulatory or inhibitory effect on MKP activity (e.g., MKP gene expression) as identified by a screening assay described herein can be administered to individuals to treat (prophylactically or therapeutically) MKP-associated disorders (e.g., aberrant or deficient metabolic function or expression) associated with aberrant or unwanted MKP activity. In conjunction with such treatment, pharmacogenomics (i.e., the study of the relationship between an individual's genotype and that individual's response to a foreign compound or drug) can be considered. Differences in metabolism of therapeutics can lead to severe toxicity or therapeutic failure by altering the relation between dose and blood concentration of the pharmacologically active drug. Thus, a physician or clinician can consider applying knowledge obtained in relevant pharmacogenomics studies in determining whether to administer a MKP molecule or MKP modulator as well as tailoring the dosage and/or therapeutic regimen of treatment with a MKP molecule or MKP modulator.

Pharmacogenomics deals with clinically significant hereditary variations in the response to drugs due to altered drug disposition and abnormal action in affected persons. See, for example, Eichelbaum et al. (1996) Clin. Exp. Pharmacol. Physiol. 23:983-985 and Linder et al. (1997) Clin. Chem. 43:254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP can occur once per every 1000 bases of DNA. A SNP can be involved in a disease process, however, the vast majority can not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that can be common among such genetically similar individuals.

Alternatively, a method termed the “candidate gene approach”, can be utilized to identify genes that predict drug response. According to this method, if a gene that encodes a drug's target is known (e.g., a MKP protein of the invention), all common variants of that gene can be fairly easily identified in the population and it can be determined if having one version of the gene versus another is associated with a particular drug response.

Alternatively, a method termed the “gene expression profiling”, can be utilized to identify genes that predict drug response. For example, the gene expression of an animal dosed with a drug (e.g., a MKP molecule or MKP modulator of the invention) can give an indication whether gene pathways related to toxicity have been turned on.

Information generated from more than one of the above pharmacogenomics approaches can be used to determine appropriate dosage and treatment regimens for prophylactic or therapeutic treatment of an individual. This knowledge, when applied to dosing or drug selection, can avoid adverse reactions or therapeutic failure and thus enhance therapeutic or prophylactic efficiency when treating a subject with a MKP molecule or MKP modulator, such as a modulator identified by one of the exemplary screening assays described herein.

The invention further provides methods for identifying new agents, or combinations, that are based on identifying agents that modulate the activity of one or more of the gene products encoded by one or more of the MKP genes of the invention, wherein these products can be associated with resistance of the cells to a therapeutic agent. Specifically, the activity of the proteins encoded by the MKP genes of the invention can be used as a basis for identifying agents for overcoming agent resistance. By blocking the activity of one or more of the resistance proteins, target cells, e.g., human cells, will become sensitive to treatment with an agent to which the unmodified target cells were resistant.

Monitoring the influence of agents (e.g., drugs) on the expression or activity of a MKP protein can be applied in clinical trials. For example, the effectiveness of an agent determined by a screening assay as described herein to increase MKP gene expression, protein levels, or upregulate MKP activity, can be monitored in clinical trials of subjects exhibiting decreased MKP gene expression, protein levels, or downregulated MKP activity. Alternatively, the effectiveness of an agent determined by a screening assay to decrease MKP gene expression, protein levels, or downregulate MKP activity, can be monitored in clinical trials of subjects exhibiting increased MKP gene expression, protein levels, or upregulated MKP activity. In such clinical trials, the expression or activity of a MKP gene, and preferably, other genes that have been implicated in, for example, an MKP-associated disorder can be used as a “read out” or markers of the phenotype of a particular cell.

Other Embodiments

In another aspect, the invention features a method of analyzing a plurality of capture probes. The method is useful, e.g., to analyze gene expression. The method includes: providing a two dimensional array having a plurality of addresses, each address of the plurality being positionally distinguishable from each other address of the plurality, and each address of the plurality having a unique capture probe, e.g., a nucleic acid or peptide sequence, wherein the capture probes are from a cell or subject which expresses MKP or from a cell or subject in which a MKP mediated response has been elicited; contacting the array with a MKP nucleic acid (preferably purified), a MKP polypeptide (preferably purified), or an anti-MKP antibody, and thereby evaluating the plurality of capture probes. Binding, e.g., in the case of a nucleic acid, hybridization with a capture probe at an address of the plurality, is detected, e.g., by a signal generated from a label attached to the MKP nucleic acid, polypeptide, or antibody.

The capture probes can be a set of nucleic acids from a selected sample, e.g., a sample of nucleic acids derived from a control or non-stimulated tissue or cell.

The method can include contacting the MKP nucleic acid, polypeptide, or antibody with a first array having a plurality of capture probes and a second array having a different plurality of capture probes. The results of each hybridization can be compared, e.g., to analyze differences in expression between a first and second sample. The first plurality of capture probes can be from a control sample, e.g., a wild type, normal, or non-diseased, non-stimulated, sample, e.g., a biological fluid, tissue, or cell sample. The second plurality of capture probes can be from an experimental sample, e.g., a mutant type, at risk, disease-state or disorder-state, or stimulated, sample, e.g., a biological fluid, tissue, or cell sample.

The plurality of capture probes can be a plurality of nucleic acid probes each of which specifically hybridizes, with an allele of MKP. Such methods can be used to diagnose a subject, e.g., to evaluate risk for a disease or disorder, to evaluate suitability of a selected treatment for a subject, to evaluate whether a subject has a disease or disorder.

The method can be used to detect SNPs, as described above.

In another aspect, the invention features, a method of analyzing MKP, e.g., analyzing structure, function, or relatedness to other nucleic acid or amino acid sequences. The method includes: providing a MKP nucleic acid or amino acid sequence; comparing the MKP sequence with one or more preferably a plurality of sequences from a collection of sequences, e.g., a nucleic acid or protein sequence database; to thereby analyze MKP.

The method can include evaluating the sequence identity between a MKP sequence and a database sequence. The method can be performed by accessing the database at a second site, e.g., over the internet. Preferred databases include GenBank™ and SwissProt.

In another aspect, the invention features, a set of oligonucleotides, useful, e.g., for identifying SNP's, or identifying specific alleles of MKP. The set includes a plurality of. oligonucleotides, each of which has a different nucleotide at an interrogation position, e.g., an SNP or the site of a mutation. In a preferred embodiment, the oligonucleotides of the plurality identical in sequence with one another (except for differences in length). The oligonucleotides can be provided with differential labels, such that an oligonucleotide which hybridizes to one allele provides a signal that is distinguishable from an oligonucleotides which hybridizes to a second allele.

The sequences of MKP molecules are provided in a variety of mediums to facilitate use thereof. A sequence can be provided as a manufacture, other than an isolated nucleic acid or amino acid molecule, which contains a MKP molecule. Such a manufacture can provide a nucleotide or amino acid sequence, e.g., an open reading frame, in a form which allows examination of the manufacture using means not directly applicable to examining the nucleotide or amino acid sequences, or a subset thereof, as they exist in nature or in purified form.

A MKP nucleotide or amino acid sequence can be recorded on computer readable media. As used herein, “computer readable media” refers to any medium that can be read and accessed directly by a computer. Such media include, but are not limited to: magnetic storage media, such as floppy discs, hard disc storage medium, and magnetic tape; optical storage media such as compact disc and CD-ROM; electrical storage media such as RAM, ROM, EPROM, EEPROM, and the like; and general hard disks and hybrids of these categories such as magnetic/optical storage media. The medium is adapted or configured for having thereon MKP sequence information of the invention.

As used herein, the term “electronic apparatus” is intended to include any suitable computing or processing apparatus of other device configured or adapted for storing data or information. Examples of electronic apparatus suitable for use with the invention include stand-alone computing apparatus; networks, including a local area network (LAN), a wide area network (WAN) Internet, Intranet, and Extranet; electronic appliances such as personal digital assistants (PDAs), cellular phones, pagers, and the like; and local and distributed processing systems.

As used herein, “recorded” refers to a process for storing or encoding information on the electronic apparatus readable medium. Those skilled in the art can readily adopt any of the presently known methods for recording information on known media to generate manufactures comprising the MKP sequence information.

A variety of data storage structures are available to a skilled artisan for creating a computer readable medium having recorded thereon a MKP nucleotide or amino acid sequence of the invention. The choice of the data storage structure will generally be based on the means chosen to access the stored information. In addition, a variety of data processor programs and formats can be used to store the nucleotide sequence information of the invention on computer readable medium. The sequence information can be represented in a word processing text file, formatted in commercially-available software such as WordPerfect and Microsoft Word, or represented in the form of an ASCII file, stored in a database application, such as DB2, Sybase, Oracle, or the like. The skilled artisan can readily adapt any number of data processor structuring formats (e.g., text file or database) in order to obtain computer readable medium having recorded thereon the nucleotide sequence information of the invention.

By providing the MKP nucleotide or amino acid sequences of the invention in computer readable form, the skilled artisan can routinely access the sequence information for a variety of purposes. For example, one skilled in the art can use the nucleotide or amino acid sequences of the invention in computer readable form to compare a target sequence or target structural motif with the sequence information stored within the data storage means. A search is used to identify fragments or regions of the sequences of the invention which match a particular target sequence or target motif.

The invention therefore provides a medium for holding instructions for performing a method for determining whether a subject has an MKP-associated or another metabolic disease or disorder or a pre-disposition to an MKP-associated disease or disorder, wherein the method comprises the steps of determining MKP sequence information associated with the subject and based on the MKP sequence information, determining whether the subject has a MKP-associated disease or disorder and/or recommending a particular treatment for the disease, disorder, or pre-disease condition.

The invention further provides in an electronic system and/or in a network, a method for determining whether a subject has an MKP-associated disease or disorder or a pre-disposition to a disease associated with MKP, wherein the method comprises the steps of determining MKP sequence information associated with the subject, and based on the MKP sequence information, determining whether the subject has an MKP-associated disease or disorder or a pre-disposition to an MKP-associated disease or disorder, and/or recommending a particular treatment for the disease, disorder, or pre-disease condition. The method may further comprise the step of receiving phenotypic information associated with the subject and/or acquiring from a network phenotypic information associated with the subject.

The invention also provides in a network, a method for determining whether a subject has an MKP-associated disease or disorder or a pre-disposition to an MKP-associated disease or disorder, said method comprising the steps of receiving MKP sequence information from the subject and/or information related thereto, receiving phenotypic information associated with the subject, acquiring information from the network corresponding to MKP and/or corresponding to an MKP-associated disease or disorder, and based on one or more of the phenotypic information, the MKP information (e.g., sequence information and/or information related thereto), and the acquired information, determining whether the subject has an MKP-associated disease or disorder or a pre-disposition to an MKP-associated disease or disorder. The method may further comprise the step of recommending a particular treatment for the disease, disorder, or pre-disease condition.

The invention also provides a business method for determining whether a subject has an MKP-associated disease or disorder or a pre-disposition to an MKP-associated disease or disorder, said method comprising the steps of receiving information related to MKP (e.g., sequence information and/or information related thereto), receiving phenotypic information associated with the subject, acquiring information from the network related to MKP and/or related to an MKP-associated disease or disorder, and based on one or more of the phenotypic information, the MKP information, and the acquired information, determining whether the subject has an MKP-associated disease or disorder or a pre-disposition to an MKP-associated disease or disorder. The method may further comprise the step of recommending a particular treatment for the disease, disorder, or pre-disease condition.

The invention also includes an array comprising MKP sequences of the invention. The array can be used to assay expression of one or more genes in the array. In one embodiment, the array can be used to assay gene expression in a tissue to ascertain tissue specificity of genes in the array. In this manner, up to about 7600 genes can be simultaneously assayed for expression, one of which can be MKP. This allows a profile to be developed showing a battery of genes specifically expressed in one or more tissues.

In addition to such qualitative information, the invention allows the quantitation of gene expression. Thus, not only tissue specificity, but also the level of expression of a battery of genes in the tissue if ascertainable. Thus, genes can be grouped on the basis of their tissue expression per se and level of expression in that tissue. This is useful, for example, in ascertaining the relationship of gene expression in that tissue. Thus, one tissue can be perturbed and the effect on gene expression in a second tissue can be determined. In this context, the effect of one cell type on another cell type in response to a biological stimulus can be determined. In this context, the effect of one cell type on another cell type in response to a biological stimulus can be determined. Such a determination is useful, for example, to know the effect of cell-cell interaction at the level of gene expression. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the array can be used to monitor the time course of expression of one or more genes in the array. This can occur in various biological contexts, as disclosed herein, for example development of an MKP-associated disease or disorder, progression of metabolic or another MKP-associated disease or disorder, and processes, such a cellular transformation associated with the metabolic or another MKP-associated disease or disorder.

The array is also useful for ascertaining the effect of the expression of a gene on the expression of other genes in the same cell or in different cells (e.g., ascertaining the effect of MKP expression on the expression of other genes). This provides, for example, for a selection of alternate molecular targets for therapeutic intervention if the ultimate or downstream target cannot be regulated.

The array is also useful for ascertaining differential expression patterns of one or more genes in normal and abnormal cells. This provides a battery of genes (e.g., including MKP) that could serve as a molecular target for diagnosis or therapeutic intervention.

As used herein, a “target sequence” can be any DNA or amino acid sequence of six or more nucleotides or two or more amino acids. A skilled artisan can readily recognize that the longer a target sequence is, the less likely a target sequence will be present as a random occurrence in the database. Typical sequence lengths of a target sequence are from about 10 to 100 amino acids or from about 30 to 300 nucleotide residues. However, it is well recognized that commercially important fragments, such as sequence fragments involved in gene expression and protein processing, may be of shorter length.

Computer software is publicly available which allows a skilled artisan to access sequence information provided in a computer readable medium for analysis and comparison to other sequences. A variety of known algorithms are disclosed publicly and a variety of commercially available software for conducting search means are and can be used in the computer-based systems of the invention. Examples of such software include, but are not limited to, MacPattern (EMBL), BLASTN and BLASTX (NCBI).

Thus, the invention features a method of making a computer readable record of a sequence of a MKP sequence which includes recording the sequence on a computer readable matrix. In a preferred embodiment the record includes one or more of the following: identification of an ORF; identification of a domain, region, or site; identification of the start of transcription; identification of the transcription terminator; the full length amino acid sequence of the protein, or a mature form thereof; the 5′ end of the translated region.

In another aspect, the invention features a method of analyzing a sequence. The method includes: providing a MKP sequence, or record, in computer readable form; comparing a second sequence to the MKP sequence; thereby analyzing a sequence. Comparison can include comparing to sequences for sequence identity or determining if one sequence is included within the other, e.g., determining if the MKP sequence includes a sequence being compared. In a preferred embodiment the MKP or second sequence is stored on a first computer, e.g., at a first site and the comparison is performed, read, or recorded on a second computer, e.g., at a second site. E.g., the MKP or second sequence can be stored in a public or proprietary database in one computer, and the results of the comparison performed, read, or recorded on a second computer. In a preferred embodiment the record includes one or more of the following: identification of an ORF; identification of a domain, region, or site; identification of the start of transcription; identification of the transcription terminator; the full length amino acid sequence of the protein, or a mature form thereof; the 5′ end of the translated region.

### EXAMPLES

### Example 1

### Insulin Resistance Expression Cloning (IREC)

The MKP molecules of the invention were discovered to play a role in metabolism (e.g., in metabolic pathways and disorders as defined herein) during insulin resistance expression cloning (IREC) experiments. Artificial constructs were manufactured by cloning PEPCK (specifically, PEPCKp600), an insulin-repressed promoter, upstream of a reporter gene, secreted alkaline phosphatase (SEAP). As described in detail herein, clones of interest (e.g., mouse MKP3 and mouse MKP4) were those which were discovered to reverse insulin's inhibitory effects on the constructs (e.g., those which could induce alkaline phosphatase activity which was previously repressed by insulin).

A cDNA expression library (“jamfa library”) was manufactured from white adipose tissue of ob/ob mice models of obesity. The artificial PEPCK constructs and single clones from the jamfa library were co-transfected into a rat hepatoma cell line (H4IIE cells) using FuGene 6 (Boehringer). The cells were split so that they are about 50-60% confluent for transfection. (55 ul/well at 34.82×10⁴/ml).

Transfection occurred the following day. The media was changed at least one hour before transfection with 150 ul/well. 125 ng/well of library cDNA and 156 ng/well of PEPCK promoter were added to a tube. The Fugene mix (3 ul of Fugene in 97 ul DMEM media) was made up in a separate tube, and 12 ul was added to the tube containing the library DNA and the promoter. The tube was mixed by gently tapping and then the mixture was allowed to sit for 15 min. All of the mix was transferred to the cells, and the mixture was left overnight to incubate.

The following day, the H4IIE cells were starved by using “starve media”, or DMEM and 0.1% FBS (at 200 ul/well). The cells were stimulated with either cyclic AMP (cAMP) and dexamethasone (Dex.); cAMP, Dex., and insulin; or with just starve media and EtOH (to ensure that the EtOH in which the Dex. was made did not have any effect). The stimulation was done in the morning and allowed to incubate until the next morning. All plates contained 3 wells of untransfected cells that received starve media containing EtOH (these were used to adjust for background control).

The next day the media was collected from the cells and spun down to remove cells. The samples were heat inactivated at 65° C. for 30 minutes (e.g., to inactivate any endogenous alkaline phosphatase), and 50 ul of the supernatant from the stimulated cell was then used for assaying with the Great EscAPE kit (Clonetech). Readings were done on the Wallac Microbeta 1450 (liquid scintillation counter) in Microflouor B Flat Bottom Plates (Dynex Tech). If a signal (positive) was detected from any of the clones, the procedure was repeated in triplicate, with 3 different stimulation media.

In the presence of cAmp and Dex., an increase in signal is seen. When insulin was added, it blocked cAmp and Dex., bringing the signal back down to background. The addition of library clones MKP3 and MKP4 each block insulin, enabling cAmp and Dex to stimulate PEPCK, once again giving a signal.

### Example 2

### Verification of MKP Enzymatic Activity

In order to verify that the mouse MKP3 and mouse MKP4 sequence used in the experiments described herein (and detected in the tissues described herein) in fact were capable of enzymatic activity, levels of phosphatase activity were measured by using an assay which employed an artificial substrate on which mouse MKP3 and mouse MKP4 is known to work, pNPP (p-nitrophenyl phosphate).

Mouse MKP4, mouse MKP3, and an empty control vector containing a puromycin-resistant gene were first transfected into a retrovirus packaging cell line, Bosc 23. At 48 hours post transfection, supernatants were collected and filtered through 0.45 μM syringe filters. 24 hours post transfection, recipient 3T3-F442A cells were seeded at a density of 2×10⁵ per 75 cm². For infection, recipient cells (preadipocyte cell line 3T3-F442A) were incubated with viral supernatant plus fresh DMEM (at a ratio of 3:1), containing a final concentration of 4 μg/ml polybrene. After a 24 hour incubation, cells were fed with fresh DMEM and allowed to grow to 80% confluency in 2-3 days. Cells were reseeded at a density of 6×10⁵ per 75 cm² for selection. Puromycin was used at 5 μg/ml for selection, and cells stably expressing MKP3 and MKP4 were confirmed by northern blot.

Secreted alkaline phosphatase (SEAP), which is commonly used as a convenient indicator of transcriptional regulation, was used to measure the phosphatase activity. To measure SEAP levels, conditioned media in which 3T3-F442A preadipocytes were grown was collected and centrifuged to get rid of extraneous floating cells. Then, the media was incubated at 65° C. for 30 minutes to inactivate any endogenous alkaline phosphatase. The unspecific substrate of phosphatase, pNPP (p-nitrophenyl phosphate), was added to the assay buffer and the plate was read at 405 nM. As the volume of crude lysate assayed rose, the measured enzymatic activity of MKP3 and MKP4 rose as well, and at every volume assayed, the MKP3- and MKP4-transfected retroviral vectors showed more dephosphorylation than the empty vectors. Glucose-6-phosphate phosphatase (G6P) was used as a positive control.

### Example 3

### Mouse Tissue Preparation and Analysis Genetic Obesity Paradigms

3 male C57BL/6J ob/ob, db/db, Ay and tub/tub mice and 5 lean littermate controls were received from Jackson labs at 4 weeks of age, and housed individually on normal mouse chow (West, D. B., 1992, Am. J. Physiol. 262:R1025-R1032) for 1 week prior to the initiation of the study. The four groups of 8 mice each were then sacrificed by CO₂ euthanasia and tissues were collected.

Tissue Collection and RNA Isolation

Following CO₂ asphyxiation, tissues were removed and quick frozen in liquid nitrogen. Total cellular RNA was extracted from tissue with RNAzol, according to the manufacturer's instructions. Briefly, the tissue was solubilized in an appropriate amount of RNAzol, and RNA was extracted by the addition of 1/10 v/v chloroform to the solubilized sample followed by vigorous shaking for approximately 15 seconds. The mixture was then centrifuged for 15 minutes at 12,000 g and the aqueous phase was removed to a fresh tube. RNA was precipitated with isopropanol. The resultant RNA pellet was dissolved in water.

Northern Blot Analyses

The Northern blots analyses described herein were performed as follows: RNA samples were electrophoresed in a denaturing agarose gel containing 1-1.5% agarose (SeaKem™ LE, FMC BioProducts, Rockland, Me.) containing 6.3% formaldehyde. Samples containing 5-20 μg of total RNA were mixed with denaturing loading solution (72% deionized formamide and bromophenol blue) and heated to 70° C. for 5 minutes. Samples were placed on ice and immediately loaded onto gels. Gels were run in 1×MOPS buffer (100 mM MOPS, 25 mM sodium acetate, 5 mM EDTA). After electrophoresis, the gels were stained with ethidium bromide and visualized with ultraviolet light.

After completion of electrophoresis, gels were soaked in 50 mM sodium hydroxide with gentle agitation for approximately 30 minutes to lightly cleave RNA. Gels were rinsed twice in water and then neutralized by soaking in 0.1M Tris-HCl (pH 7.5) for approximately 30 minutes. Gels were briefly equilibrated with 20×SSC (3M sodium chloride, 0.3M sodium citrate) and then transferred to nylon membranes such as Hybond™, -N, (Amersham, Inc., Arlington Heights, Ill.) or Zeta-Probe (Bio-Rad, Inc., Hercules, Calif.) overnight in 20×SSC. Membranes containing transferred RNA were baked at 80° C. for 2 hours to immobilize the RNA.

DNA fragments to be used as probes were of various sizes and were labeled using a random hexamer labeling technique. Briefly, 25 ng of a purified DNA fragment was used to generate each probe. Fragments were added to a 20 μl random hexanucleotide labeling reaction (Boehringer Mannhein, Inc., Indianapolis, Ind.) containing random hexamers and a mix of the nucleotides dCTP, dGTP, and dTTP (at a final concentration of 25 μM each). The reaction mix was heat-denatured at 100° C. for 10 minutes and then chilled on ice. 5 μl of α-³²P-dATP (50 μCi; Amersham, Inc., Arlington Heights, Ill.) and Klenow DNA polymerase (2 units; Boehringer Mannheim, Inc., Indianapolis, Ind.) were added. Reactions were incubated at 37° for 30 minutes. Following incubation, 30 μl water was added to the labeling reaction and unincorporated nucleotides were removed by passing the reactions through a BioSpin-6™ chromatography column (Bio-Rad, Inc., Hercules, Calif.). Specific incorporation was determined using a scintillation counter. 1-5×10⁶ cpm were used per ml hybridization mixture.

Nylon membranes containing immobilized RNA were prehybridized according to manufacturer's instructions. Radiolabelled probes were heat denatured at 70° C. in 50% deionized formamide for 10 minutes and ten added to the hybridization mixture (containing 50% formamide, 10% dextran sulfate, 0.1% SDS, 100 μg/ml sheared salmon sperm DNA, 5×SSC, 5×Denhardt's solution, 30 mM Tris-HCl (pH 8.5), 50 mM NaPO₄ (pH 6.5). Hybridizations were carried out at 42° C. overnight. Nylon membranes were then bathed for 2 minutes in a wash solution of 0.2×SSC and 0.1% SDS at room temperature to remove most of the remaining hybridization solution. The membranes were then bathed twice in fresh 42° C. preheated wash solution for 20 minutes. Filters were covered in plastic wrap and exposed to autoradiographic film to visualize results.

Northern blots on which mouse MKP3 and mouse MKP4 mRNA were run show expression in a variety of tissues. Mouse MKP3 is highly expressed in kidney, brown adipose tissue (BAT), lung, and intestine, expressed at moderate levels in testis and white adipose tissue (WAT), heart, and spleen, and expressed at lower levels in all other tissues tested. Mouse MKP4 is expressed at almost equal levels in white adipose tissue (WAT), kidney, and testis. Normally, mouse MKP4 is undetectable in brown adipose tissue (BAT), liver, and muscle, but can be detected in those tissues in the obese state.

Mouse MKP3 and mouse MKP4 are expressed at elevated levels in white adipose tissue (WAT) and brown adipose tissue (BAT) of ob/ob and db/db mice models of obesity, as well as of diet induced obesity mouse models (A^{Y}, tub/+, tub/tub, and HFD), as compared to the same tissues in wild type mice. In all cases, the difference in expression levels between the wild type mice tissues and the obesity model mice tissues was more marked in the ob/ob and db/db models than with the others (e.g., A^{Y}, tub/+, and tub/tub)(i.e., the MKP3 and MKP4 upregulation in obesity models was more pronounced in ob/ob and db/db).

The expression level of mouse MKP3 was also elevated in liver in ob/ob and db/db nice, compared to liver of the wild type mice. And although MKP3 is expressed in muscle in both wild type and obese mice, there does not seem to be a disparity of expression, as seen with the fat tissue samples. Likewise, although MKP4 is expressed in kidney and testis of both wild type and obese mice, and to a lesser extent in muscle and liver, there does not seem to be a disparity of expression, as seen with the fat tissue samples.

Furthermore, experiments were performed to compare MKP4 expression between mice put on a high fat diet (HPD) and mice which were fed a low fat diet. The experiments were performed as follows: three week old male C57BL/6J mice were purchased from the Jackson Laboratory, and were acclimated on a standard chow diet for a week. Half of the mice were then put on a high fat diet containing 45% fat (Research Diet, Inc.), and the other half was given a low fat diet containing 10% fat (Research Diet, Inc.). Body weight was measured every two weeks until the high fat group was 22% heavier than the low fat group. The mice were then sacrificed by CO₂ asphyxiation, and white adipose tissue (WAT) was collected for RNA extraction. Northern analysis (not shown) was performed on the white adipose tissue samples from both HFD and wild type mice, confirming upregulation (much higher expression) of MKP4 in the high fat diet model mice.

Preadipocyte-Differentiation Northern Blot Analyses

In addition to being upregulated in predominantly insulin-related (e.g.insulin-responsive) tissues in mice models of obesity, MKP family members (e.g., mouse MKP4) are upregulated in preadipocytes as they differentiate. A Northern blot was performed analyzing MKP3 and MKP4 expression in preadipocytes, adipocytes, and adipocytes treated with 5 ug/ml insulin for 5 time points. The expression of MKP3 and MKP4 is stronger (e.g., the genes are upregulated) in adipocytes versus preadipocytes, although acute treatment with insulin has no effect.

The preadipocytes (in which retroviral MKP3 or MKP4 were infected) used were from the cell line 3T3-F442A, and were maintained in DMEM and supplemented with 10% bovine calf serum (BCS). To induce preadipocyte differentiation, the cells were grown to 80% confluency in DMEM and supplemented with 10% cosmic calf serum (CCS). Then, the cells were exposed to the adipogenic (i.e., differentiative) mixture of the reagents insulin (5 μg/ml), dexamethasone (1 μM), and isobutylmethylxanthine (0.5 mM) for 3 days, and then maintained in the presence of insulin until they differentiated. The cells were then cultured in serum-free DMEM for 24 hours before acute insulin treatment and Northern blot hybridization was performed for MKP3 and MKP4 expression. The cells were harvested at different time points relative to their exposure to the adipogenic agents, and probed for mouse MKP3 and/or mouse MKP4 expression.

The results seen in the Northern blot show that expression levels of MKP3 and MKP4 increase with adipocyte differentiation, although acute treatment with insulin has no effect. In particular, the levels of MKP3 and MKP4 expression increased as follows: preadipocytes; adipocytes; adipocytes treated with insulin for 30 minutes; adipocytes treated with insulin for one hour: adipocytes treated with insulin for three hours; and adipocytes treated with insulin for five hours. Increased expression levels ceased after five hours' worth of expression, and the adipocytes treated with insulin for ten hours revealed MKP3 and MKP4 expression levels which were lower than at least those seen with adipocytes treated with insulin for five hours.

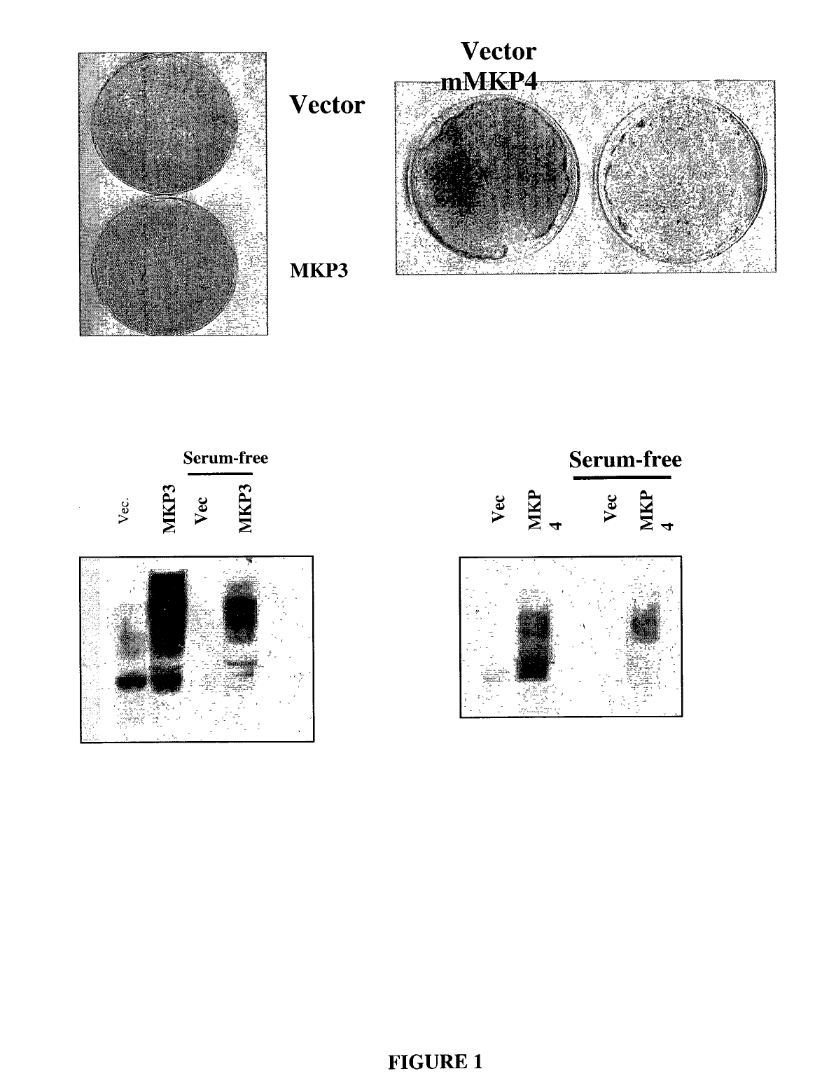

As seen in FIG. 1, overexpression of MKP family members (e.g., mouse MKP4) can inhibit preadipocyte differentiation (i.e., prevent them from becoming mature fat cells (adipocytes)). Oil red O stain works on lipid droplets, of which there are more in adipocytes than their precursors. Less stain is noticeable in the preadipocytes in which mouse MKP4 is overexpressed, as the phosphatase kinase has prevented the maturation of the cells. To perform the experiment, the preadipocyte cell line 3T3-F442A was maintained in DMEM and supplemented with 10% bovine calf serum (BCS). To induce differentiation, the cells were grown to 80% confluency in DMEM and supplemented with 10% cosmic calf serum (CCS). Then, the cells were exposed to adipogenic reagents for 2-3 days (5 μg/ml insulin, 1 μM Dexamethasone and 0.5 mM isobutylmethylxanthine). After the 2-3 day induction period, the cells were maintained in DMEM, and supplemented with 10% CCS and 5 μg/ml insulin until the cells fully differentiated (which usually takes about 10 to 14 days). To compare differentiation, cells were stained with oil red O.

### Example 4

### Overexpression of MKP4 Inhibits Insulin Stimulated Glucose Uptake

Cultured preadipocytes were induced to differentiate into mature adipocytes via means described supra. Then, the cells were infected by either vector (control) or MKP4-expressing adenovirus. Forty eight hours after infection, cells were deprived of serum. Seventy-two hours after infection, cells were stimulated with insulin and glucose uptake was determined by incubating cells with 3H-labeled deoxy glucose, lysing the cells, and measuring the lysate for radioactivity. The radioactivity counts show that the amount of glucose uptaken by insulin-stimulated MKP4 expressing cells is 30% less than that of vector only-expressing cells.

The contents of all references, patents and published patent applications cited throughout this application are incorporated herein by reference.

Equivalents

The invention is not to be limited in scope by the specific embodiments described herein, which are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Those skilled in the art will recognize, or be able to ascertain using no more than routine experimentation, many equivalents to the specific embodiments of the invention described herein.

What is claimed is:

1. A method for treating or preventing a metabolic disorder in a subject, said disorder characterized by aberrant MKP polypeptide activity or aberrant MKP nucleic acid expression, comprising administering to the subject an MKP modulator in amounts effective for treating or preventing the metabolic disorder.

2. The method of claim 1, wherein the MKP modulator is a small molecule.

3. The method of claim 1, wherein the MKP modulator is a nucleic acid molecule that is antisense to MKP-encoding nucleic acid molecules.

4. The method of claim 1, wherein the MKP modulator is an anti-MKP antibody.

5. The method of claim 1, wherein the MKP modulator is a MKP polypeptide, or a fragment thereof.

6. The method of claim 1, wherein the MKP modulator is capable of modulating MKP nucleic acid expression.

7. The method of any of claims 2 to 6, wherein said MKP modulator is administered in a pharmaceutically acceptable formulation.

8. The method of claim 1, wherein the disorder is a disorder associated with aberrant modulation of glycogen synthesis.

9. The method of claim 1, wherein the metabolic disorder is a disorder associated with aberrant cellular utilization of energy sources.

10. The method of claim 9, wherein the metabolic disorder is obesity.

11. The method of claim 9, wherein the metabolic disorder is diabetes.

12. A method for identifying a compound implicated in a metabolic disorder and capable of modulating an MKP activity comprising:
a) contacting a sample derived from a patient afflicated with or at risk for a metabolic disorder with a test compound; and
b) assaying the ability of the test compound to modulate the expression of a MKP nucleic acid or the activity of a MKP polypeptide;
thereby identifying a compound implicated in a metabolic disorder and capable of modulating an MKP activity.

13. The method of claim 12, wherein the test compound is capable of selectively binding to an MKP polypeptide.

14. The method of claim 12, wherein the test compound is an antibody.

15. The method of claim 12, wherein the test compound is a nucleic acid molecule that is antisense to MKP-encoding nucleic acid molecules.

16. The method of claim 12, wherein the test compound is a small molecule.

17. A method for modulating gene products involved in body weight disorders or with gene products which are relevant to appetite and body weight regulation comprising administering to the subject an MKP modulator, in amounts effective for modulating said gene products.

18. The method of claim 17, wherein the MKP modulator is a small molecule.

19. The method of claim 17, wherein the MKP modulator is a nucleic acid molecule that is antisense to MKP-encoding nucleic acid molecules.

20. The method of claim 17, wherein the MKP modulator is an anti-MKP antibody.

21. The method of claim 17, wherein the MKP modulator is a MKP polypeptide, or a fragment thereof.

22. The method of claim 17, wherein the MKP modulator is capable of modulating MKP nucleic acid expression.

23. The method of any of claims 18 to 22, wherein said MKP modulator is administered in a pharmaceutically acceptable formulation.

24. A method for modulating the effects of insulin on metabolism in a subject, comprising administering to the subject an MKP modulator, in amounts effective for modulating the effects of insulin.

25. The method of claim 24, wherein the MKP modulator is a small molecule.

26. The method of claim 24, wherein the MKP modulator is a nucleic acid molecule that is antisense to MKP-encoding nucleic acid molecules.

27. The method of claim 24, wherein the MKP modulator is an anti-MKP antibody.

28. The method of claim 24, wherein the MKP modulator is a MKP polypeptide, or a fragment thereof

29. The method of claim 24, wherein the MKP modulator is capable of modulating MKP nucleic acid expression.

30. The method of any of claims 25 to 29, wherein said MKP modulator is administered in a pharmaceutically acceptable formulation.

31. A method for modulating the effects of glucose on metabolism in a subject, comprising administering to the subject an MKP modulator, in amounts effective for modulating the effects of glucose.

32. The method of claim 31, wherein the MKP modulator is a small molecule.

33. The method of claim 31, wherein the MKP modulator is a nucleic acid molecule that is antisense to MKP-encoding nucleic acid molecules.

34. The method of claim 31, wherein the MKP modulator is an anti-MKP antibody.

35. The method of claim 31, wherein the MKP modulator is a MKP polypeptide, or a fragment thereof

36. The method of claim 31, wherein the MKP modulator is capable of modulating MKP nucleic acid expression.

37. The method of any of claims 32 to 36, wherein said MKP modulator is administered in a pharmaceutically acceptable formulation.

38. A method for modulating differentiation of adipocytes in a subject, comprising administering to the subject an MKP modulator, in amounts effective for modulating adipocyte differentiation.

39. The method of claim 38, wherein the MKP modulator is a small molecule.

40. The method of claim 38, wherein the MKP modulator is a nucleic acid molecule that is antisense to MKP-encoding nucleic acid molecules.

41. The method of claim 38, wherein the MKP modulator is an anti-MKP antibody.

42. The method of claim 38, wherein the MKP modulator is a MKP polypeptide, or a fragment thereof

43. The method of claim 38, wherein the MKP modulator is capable of modulating MKP nucleic acid expression.

44. The method of any of claims 39 to 43, wherein said MKP modulator is administered in a pharmaceutically acceptable formulation.

45. An isolated nucleic acid molecule selected from the group consisting of:
a. a nucleic acid molecule comprising a nucleotide sequence which is at least 98% identical to the nucleotide sequence of SEQ ID NO:1 or SEQ ID NO:3; and
b. a nucleic acid molecule which encodes a polypeptide comprising the amino acid sequence of SEQ ID NO:2.

46. An isolated nucleic acid molecule selected from the group consisting of:
a. a nucleic acid molecule comprising a nucleotide sequence which is at least 75% identical to the nucleotide sequence of SEQ ID NO:4 or SEQ ID NO:6;
b. a nucleic acid molecule comprising a fragment of at least 300 nucleotides of the nucleotide sequence of SEQ ID NO:4 or SEQ ID NO:6;
c. a nucleic acid molecule which encodes a polypeptide comprising the amino acid sequence of SEQ ID NO:5;
d. a nucleic acid molecule which encodes a fragment of a polypeptide comprising the amino acid sequence of SEQ ID NO:5, wherein the fragment comprises at least 100 contiguous amino acids of SEQ ID NO:5; and
e. a nucleic acid molecule which encodes a naturally occurring allelic variant of a polypeptide comprising the amino acid sequence of SEQ ID NO:5, wherein the nucleic acid molecule hybridizes to a nucleic acid molecule comprising SEQ ID NO:4, 6, or a complement thereof, under stringent conditions.

47. An isolated polypeptide which is encoded by a nucleic acid molecule comprising a nucleotide sequence which is at least 98% identical to a nucleic acid comprising the nucleotide sequence of SEQ ID NO:1, SEQ ID NO:3, or a complement thereof.

48. An isolated polypeptide selected from the group consisting of:
a) a polypeptide which is encoded by a nucleic acid molecule comprising a nucleotide sequence which is at least 75% identical to a nucleic acid comprising the nucleotide sequence of SEQ ID NO:4, SEQ ID NO:6, or a complement thereof; and
b) a fragment of a polypeptide comprising the amino acid sequence of SEQ ID NO:5, wherein the fragment comprises at least 300 contiguous amino acids of SEQ ID NO:5.
